(19) United States
(12) Patent Application Publication
(10) Pub. No.: US 2008/0132522 A1
(43) Pub. Date: Jun. 5, 2008

(54) 2-Substituted Pyrimidines, Method for Their Production and Their Use for Controlling Pathogenic Fungi

(75) Inventors: Joachim Rheinheimer, Ludwigshafen (DE); Frank Schieweck, Hessheim (DE); Thomas Grote, Wachenheim (DE); Carsten Blettner, Hong Kong (CN); Anja Schwogler, Mannheim (DE); Markus Gewehr, Kastellaun (DE); Wassilios Grammenos, Ludwigshafen (DE); Udo Hunger, Mainz (DE); Bernd Muller, Frankenthal (DE); Peter Schafer, Ottersheim (DE); Jochen Dietz, Mannheim (DE); John-Bryan Speakman, Bobenheim (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 11/631,006

(22) PCT Filed: Jul. 12, 2005
(86) PCT No.: PCT/EP05/07517

§ 371 Date: Dec. 28, 2006

(30) Foreign Application Priority Data
Jul. 14, 2004 (DE) 10 2004 034 197.4

(51) Int. Cl. A01N 43/54; C07D 401/04; A01P 3/00; A01P 13/00; A01P 15/00

(52) U.S. Cl. 514/256; 544/333

(57) ABSTRACT

The invention relates to 2-substituted pyrimidines of the formula I

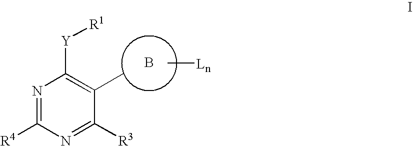

in which the index n and the substituents R¹ to R⁴, B and L are as defined in the description and
Y is a group —O— or —S—,
   and to processes for their preparation, pesticidal compositions and methods for controlling harmful fungi and animal pests using the compounds according to the invention.

The present invention relates to 2-substituted pyrimidines of the formula I

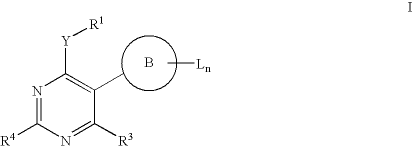

in which the indices and the substituents are as defined below:

### Y is —O— or —S—;

R¹ is C₁-C₈-alkyl, C₂-C₈-alkenyl, C₂-C₈-alkynyl, C₃-C₆-cycloalkyl, C₃-C₆-halocycloalkyl, di-C₁-C₈-alkylamino, C₁-C₈-alkylamino, where R¹ for its part may be partially or fully halogenated or may carry one to four groups R²:
R² is cyano, C₁-C₆-alkyl, C₃-C₆-cycloalkyl, C₄-C₆-cycloalkenyl, hydroxyl, C₁-C₆-alkoxy, C₂-C₈-alkenyloxy, C₂-C₈-alkynyloxy, C₃-C₆-cycloalkyloxy, C₄-C₆-cycloalkenyloxy, C₁-C₆-alkylthio, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A or phenyl, where the phenyl moiety may carry one to three radicals selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N-OA), N(A′)A; or —CH₂—Si(C₁-C₆-alkyl)₂;
R³ is halogen, cyano, azido, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di(C₁-C₆-alkyl)amino or C₁-C₆-alkylamino, where the alkyl, alkenyl and alkynyl radicals of R³ may be substituted by halogen, cyano, nitro, C₁-C₂-alkoxy or C₁-C₄-alkoxycarbonyl;
R⁴ is a five- or six-membered saturated, partially unsaturated or aromatic mono- or bicyclic heterocycle which comprises one to four heteroatoms from the group consisting of O, N and S which for its part may be partially or fully halogenated or may carry one to four groups R^{u}:
R^{u} is cyano, C₁-C₈-alkyl, C₂-C₈-alkenyl, C₂-C₈-alkynyl, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A, where m, A, A′, A″ are as defined above;
R⁴ may furthermore be:
cyano, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c},

### ON(═CR^{a}R^{b}), O—C(=Z)R^{a},

NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}, where

### Z is O, S, NR^{a}, NOR^{a} or N—NR^{z}R^{c};

R^{a}, R^{b}, R^{c} independently of one another are hydrogen, C₁-C₆-alkyl, C₂-C₆-alkenyl, C₂-C₆-alkynyl, C₃-C₆-cycloalkyl or C₄-C₆-cycloalkenyl;
R^{b′} has the same meanings as R^{b}, except for hydrogen;
R^{z} has the same meanings as R^{a} and may additionally be —CO—R^{a};
where the aliphatic or alicyclic groups of the radical definitions of R^{a}, R^{b}, R^{c} or R^{z} for their part may be partially or fully halogenated or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, and where two of the radicals R^{a}, R^{b}, R^{c} or R^{z} together with the atoms, to which they are attached, may form a five- or six-membered saturated, partially unsaturated or aromatic heterocycle which comprises one to four heteroatoms from the group consisting of O, N and S;
\textcircled{B} is a five- or six-membered hetaryl which comprises 1 to 3 heteroatoms selected from the group consisting of O, N and S or is phenyl;
n is an integer from 1 to 5;
L is halogen, cyano, cyanato (OCN), C₁-C₈-alkyl, C₂-C₈-alkenyl, C₂-C₈-alkynyl, C₁-C₆-alkoxy, C₂-C₈-alkenyloxy, C₂-C₈-alkynyloxy, C₃-C₆-cycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A,
m is 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by nitro, cyanato, cyano or C₁-C₄-alkoxy; or A and A′ together with the atoms to which they are attached are a five- or six-membered saturated, partially unsaturated or aromatic heterocycle which comprises one to four heteroatoms from the group consisting of O, N and S;
where the aliphatic groups of the radical definitions of L for their part may be partially or fully halogenated or may carry one to four groups R^{L}:
R^{L} is cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A.

2-Substituted pyrimidines having fungicidal action are already known from the literature (EP-A 407899, WO-A 02/074753 and WO-A 03/043993).

However, the activity of the abovementioned pyrimidines is in many cases unsatisfactory. Accordingly, it was an object of the present invention to provide further compounds having fungicidal action.

We have found that this object is achieved by the 2-substituted pyrimidines I defined at the outset. Moreover, we have found processes for their preparation and compositions comprising them for controlling harmful fungi and their use for this purpose.

The compounds of the formula I can be obtained by different routes.

The compounds described can be prepared, for example, from appropriately substituted phenylmalonates 2. These are known or obtainable analogously to the known substances.

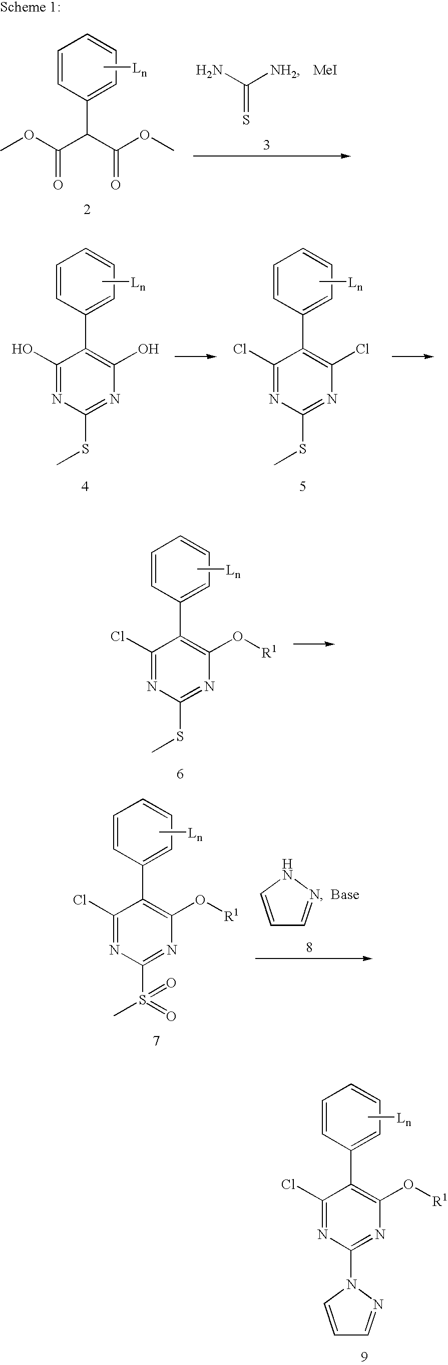

Using thiourea (3) and a methylating agent or using S-methylisothiourea, the phenylmalonates 2 can be converted into the dihydroxypyrimidine derivatives 4 (see Scheme 1). Suitable methylating agents are, for example, methyl iodide, methyl bromide or dimethyl sulfate.

It is possible to employ a solvent which is inert under the reaction conditions and in which the reactants are sufficiently soluble. The reaction temperature can be between −20° C. and 150° C. and is preferably between 0° C. and 100° C.

The dihydroxypyrimidines 4 obtainable in this manner can then be chlorinated using customary methods to give the dichloropyrimidines 5. The use of phosphorus oxychloride, if appropriate with addition of an amine such as diethylaniline, an amine hydrochloride or dimethylformamide, has been found to be particularly suitable. Usually, it is advantageous to carry out the reaction at elevated temperature to increase the conversion rate.

Dichloropyrimidines 5 can then be substituted further by different routes. It has been found that, frequently, the regioselectivity depends to an unexpected degree on the chosen co-reactants and reaction conditions. In the route shown in Scheme 1, the alcohol is attached nucleophilicly in the 2 or 4-position.

The thiolate group (C₁-C₆-alkylthio) in the 2-position of the compound 6 is oxidized to the C₁-C₆-alkylsulfonyl (C₁-C₆-alkylS[═O]₂—) group of the compound 7 and thus converted into a leaving group for further exchange reactions. Hydrogen peroxide or peracids of organic carboxylic acids have been found to be particularly suitable oxidizing agents. However, the oxidation can also be carried out using, for example, selenium dioxide.

For introducing a heterocyclic radical R⁴ into the 2-position of the compound 7 it is possible to use the heterocycle (such as, for example, pyrazole or triazole) directly, depending on its nucleophilicity. In these cases, an auxiliary base is usually employed. It is also possible to introduce heterocyclic substituents via palladium- or nickel-catalyzed reactions. In these cases, the heterocycle carries a suitable organometallic leaving group.

According to Scheme 2 it is possible to introduce, for example, cyanides (nitriles) into the 6-position of the compound 7 which can then be reacted further by known methods to give, for example, amides, amidoximes or amidines. Amidoximes 9 or 10, for example, can be prepared from the nitrites 8 and hydroxylamine or O-alkylated hydroxylamines.

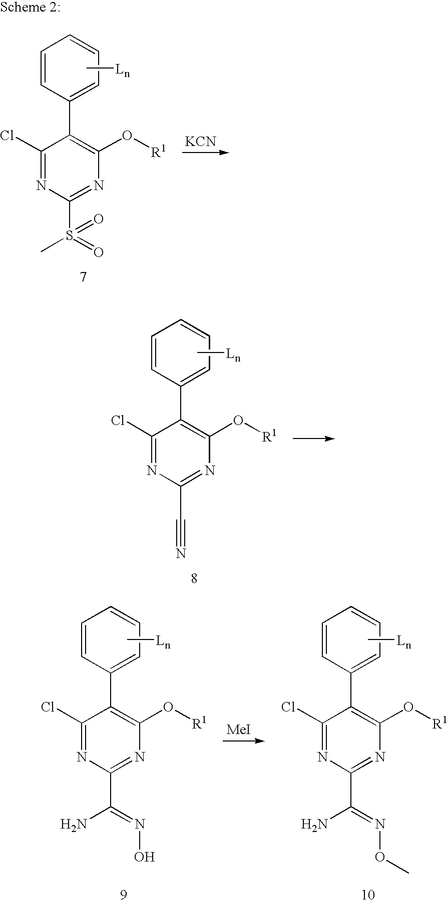

What was said above also applies, for example, to the preparation of compounds in which R³ is an alkyl group. As illustrated in more detail above, such an alkyl group (R³) can be prepared using organometallic compounds of the formula (R³)ₙ-Mⁿ where M is as defined above. If R³ is a cyano group or an alkoxy substituent, the radical R³ can be introduced by reaction with alkali metal cyanides and alkali metal alkoxides, respectively.

In the definitions of the symbols given in the formulae above, collective terms were used which are generally representative for the following substituents:

halogen: fluorine, chlorine, bromine and iodine;
alkyl and the alkyl moieties of, for example, alkoxy, alkylamino, alkoxycarbonyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 4, 6 or 8 carbon atoms (as mentioned above), where in these groups some or all of the hydrogen atoms may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6 or 8 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkadienyl: unsaturated straight-chain or branched hydrocarbon radicals having 4 to 8 carbon atoms and two double bonds in any position;
haloalkenyl: unsaturated straight-chain or branched hydrocarbon radicals having 2 to 8 carbon atoms and a double bond in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 carbon ring members, for example C₃-C₆-cycloalkyl such as cyclopropyl, cyclobutyl, cyclopentyl, cyclohexyl;
five- or six-membered saturated, partially unsaturated or aromatic heterocycle which comprises one to four heteroatoms from the group consisting of O, N and S, where the heterocycle in question may be attached via carbon or nitrogen:
5- or 6-membered heterocyclyl which comprises one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
5-membered heteroaryl which comprises one to four nitrogen atoms or one to three nitrogen atoms and/or one sulfur or oxygen atom: 5-membered heteroaryl groups which, in addition to carbon atoms, may comprise one to four nitrogen atoms or one to three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4,-thiadiazol-2-yl and 1,3,4-triazol-2-yl;
6-membered heteroaryl which comprises one to three or one to four nitrogen atoms: 6-membered heteroaryl groups which, in addition to carbon atoms, may comprise one to three or one to four nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The scope of the present invention includes the (R) and (S) isomers and the racemates of compounds of the formula I having chiral centers.

Hereinbelow, the embodiments of the invention are described in more detail.

With a view to the intended use of the pyrimidines of the formula I, particular preference is given to the following meanings of the substituents, in each case on their own or in combination:

Preference is given to compounds I in which R¹ is C₁-C₆-alkyl, C₁-C₆-haloalkyl, C₂-C₆-alkenyl, C₂-C₆-alkynyl or C₃-C₆-cycloalkyl.

Especially preferred are compounds I in which R¹ is C₁-C₆-haloalkyl, C₂-C₆-alkenyl or C₁-C₆-alkyl branched in the α-position. In addition, preference is given to compounds I in which R¹ is C₁-C₄-haloalkyl.

Particularly preferred are compounds I in which Y=O and R¹ is ethyl, propyl, isopropyl, 1,2-dimethylpropyl, 1,2,2-trimethylpropyl, 1-methyl-2,2,2-trifluoroethyl or 2,2,2-trifluoroethyl.

Preference is furthermore given to those compounds I in which Y=S and R¹ is methyl, ethyl, propyl, isopropyl, 1,2-dimethylpropyl, 1,2,2-trimethylpropyl, 1-methyl-2,2,2-trifluoroethyl or 2,2,2-trifluoroethyl.

Particularly preferred are also compounds I in which R³ is C₁-C₄-alkyl which may be substituted by halogen.

Moreover, particular preference is given to compounds I in which R³ is halogen, cyano, C₁-C₄-alkyl or C₁-C₄-alkoxy.

Especially preferred are compounds I in which R³ is methyl, cyano, methoxy or, in particular, chlorine.

Preference is furthermore given to compounds I in which R⁴ is pyrrole, pyrazole, imidazole, 1,2,3-triazole, 1,2,4-triazole, tetrazole, oxazole, isoxazole, 1,3,4-oxadiazole, furan, thiophene, thiazole, isothiazole, pyridine, pyrimidine, pyrazine, pyridazine, 1,2,3-triazine, 1,2,4-triazine, 1-pyridin(1,2,-dihydro)-2-one or 1-pyrrolidone, where the heterocycle may be attached to the pyrimidine ring via C or N and may carry up to three substituents R^{u}. This preference applies both in combination with the broad definition of R^{u} given in claim 1 and with the narrower definition of R^{u} below: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N-OA), N(A′)A, N(A′)-C(═O)-A.

Particularly preferred are compounds I in which R⁴ is 1-pyrazolyl, 1-[1,2,4]triazolyl, 2-thiazolyl, 2-pyridinyl, 2-pyrimidinyl, 3-pyridazinyl, 1-pyridin(1,2-dihydro)-2-onyl or 1-pyrrolidonyl. This preference applies both in combination with the broad definition of R^{u} given in claim 1 and with the narrower definition of R^{u} below: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N-OA), N(A′)A, N(A′)-C(═O)-A.

Preference is furthermore given to compounds I in which R⁴ is pyrazolyl or [1,2,4]triazolyl.

Especially preferred are compounds I in which R⁴ is 2-pyrimidinyl. This preference applies both in combination with the broad definition of R^{u} given in claim 1 and with the narrower definition of R^{u} below: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N-OA), N(A′)A, N(A′)-C(═O)-A.

Preference is also given to compounds I in which R⁴ is cyano, C(═O)NR^{z}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CR^{c}R^{b}) or NR^{z}—OR^{a}.

Moreover, preference is given to compounds I in which R⁴ is C(=Z)OR^{a}, C(=Z)NR^{z}R^{b} or C(=Z)R^{a} and Z is O, NR^{a} or NOR^{a}.

Especially preferred are compounds I in which R⁴ is C(═O)NR^{z}R^{b} or C(═N—OCH₃)NR^{z}R^{b}.

Preference is furthermore given to compounds I in which R⁴ is C(═NH)NR^{z}R^{b} and R^{z} is an acyl substituent: —CO—Ra.
\textcircled{B} is five- or six-membered hetaryl which comprises 1 to 3 heteroatoms selected from the group consisting of O, N and S or is phenyl. Preferably, \textcircled{B} may be five- or six-membered hetaryl which comprises 1 to 3 heteroatoms selected from O, N and S, for example
   5-membered heteroaryl which comprises one to three nitrogen atoms or one to two nitrogen atoms and/or one sulfur or oxygen atom: 5-membered heteroaryl groups which, in addition to carbon atoms, may comprise one to three nitrogen atoms or one to two nitrogen atoms and/or one sulfur or oxygen atom as ring members, for example furyl, thienyl, pyrrolyl, pyrazolyl, imidazolyl, triazolyl (1,2,3-; 1,2,4-triazolyl), oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4,-thiadiazol-2-yl and 1,3,4-triazol-2-yl;
   6-membered heteroaryl which comprises one to three nitrogen atoms: 6-membered heteroaryl groups which, in addition to carbon atoms, may comprise one to three nitrogen atoms as ring members, for example pyridine, pyrimidine, pyrazine, pyridazine, 1,2,3-triazine, 1,2,4-triazine, 1,3,5-triazine, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.
\textcircled{B} is particularly preferably five-membered hetaryl which comprises 1 to 3 heteroatoms selected from the group consisting of O, N and S or is pyridyl or particularly preferably phenyl.

Especially preferred are pyrimidines I where the substituents L (L¹ to L⁵) are as defined below:
L is halogen, cyano, methyl, methoxy, —C(═O)—O-A, —C(═O)—N(A′)A, C(A′)(═N-OA), N(A′)A, N(A′)-C(═O)-A,
   A,A′ independently of one another are hydrogen, C₁-C₆-alkyl, C₂-C₆-alkenyl, C₂-C₆-alkynyl, phenyl, where the organic radicals may be partially or fully halogenated or may be substituted by C₁-C₄-alkoxy; or A and A′ together with the atoms to which they are attached are a five- or six-membered saturated heterocycle which comprises one or two heteroatoms from the group consisting of O, N and S.

Moreover, preference is given to pyrimidines I in which the group B substituted by Lₙ is

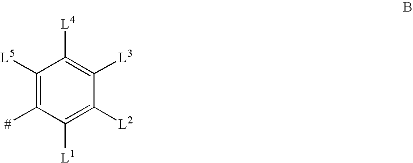

in which # is the point of attachment to the pyrimidine skeleton and
L¹ is fluorine, chlorine, CH₃ or CF₃;

L²,L⁴ independently of one another are hydrogen, CH₃ or fluorine;

L³ is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L⁵ is hydrogen, fluorine, chlorine or CH₃.

Preference is furthermore given to 2-substituted pyrimidines of the formula I′

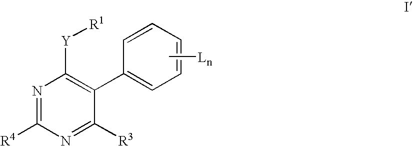

where
Y is a group —S— or —O—;
R¹ is C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, or (C₁-C₆-alkyl)C₃-C₆-cycloalkyl;
R³ is halogen, cyano, C₁-C₄-alkyl, C₁-C₄-alkoxy or C₁-C₄-haloalkyl;
R⁴ is pyrrole, pyrazole, imidazole, 1,2,3-triazole, 1,2,4-triazole, tetrazole, oxazole, isoxazole, 1,3,4-oxadiazole, furan, thiophene, thiazole, isothiazole, pyridine, pyrimidine, pyrazine, pyridazine, 1,2,3-triazine, 1,2,4-triazine, 1-pyridin(1,2-dihydro)-2-one or 1-pyrrolidone, where the heterocycle may be attached via C or N to the pyrimidine ring and may carry up to three substituents R^{u};
   R^{u} is halogen, cyano, C₁-C₈-alkyl, C₁-C₆-alkoxy, —C(═O)-A, —C(═O)—O-A, —C(═O)—N(A′)A, C(A′)(═N-OA), N(A′)A, N(A′)-C(═O)-A,
      or
      cyano, C(═O)NR^{z}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CR^{c}R^{b}), NR^{a}R^{b′} or NR^{z}—OR^{a};
n is an integer from 1 to 3 where at least one substituent L is located in the ortho-position on the phenyl ring;
L is halogen, cyano, methyl, methoxy, —C(═O)—O-A, —C(═O)—N(A′)A, C(A′)(═N-OA), N(A′)A, N(A′)-C(═O)-A,
   A,A′ independently of one another are hydrogen, C₁-C₆-alkyl, C₂-C₆-alkenyl, C₂-C₆-alkynyl, phenyl, where the organic radicals may be partially or fully halogenated or may be substituted by C₁-C₄-alkoxy; or A and A′ together with the atoms to which they are attached are a five- or six-membered saturated heterocycle which comprises one or two heteroatoms from the group consisting of O, N and S;
      where the aliphatic groups of the radical definitions of L for their part may be partially or fully halogenated.

Preference is furthermore given to 2-substituted pyrimidines of the formula I′

where
Y is a group —S— or —O—;
R¹ is C₁-C₆-alkyl, C₂-C₆-alkenyl, C₂-C₆-alkynyl, C₁-C₆-haloalkyl, C₂-C₆-haloalkenyl or C₂-C₆-haloalkynyl;
R³ is halogen, cyano, C₁-C₄-alkyl, C₁-C₄-alkoxy or C₁-C₄-haloalkyl;
R⁴ is pyrrole, pyrazole, imidazole, 1,2,3-triazole, 1,2,4-triazole, tetrazole, oxazole, isoxazole, 1,3,4-oxadiazole, furan, thiophene, thiazole, isothiazole, pyridine, pyrimidine, pyrazine, pyridazine, 1,2,3-triazine, 1,2,4-triazine, 1-pyridin(1,2-dihydro)-2-one or 1-pyrrolidone, where the heterocycle may be attached via C or N to the pyrimidine ring and may carry up to three substituents R^{u};
   R^{u} is halogen, cyano, C₁-C₈-alkyl, C₁-C₆-alkoxy, —C(═O)-A, —C(═O)—O-A, —C(═O)—N(A′)A, C(A′)(═N-OA), N(A′)A, N(A′)-C(═O)-A,
      or
      cyano, C(═O)NR^{z}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CR^{c}R^{b}) or NR^{z}—OR^{a};
n is an integer from 1 to 3 where at least one substituent L is located in the ortho-position on the phenyl ring;
L is halogen, cyano, methyl, methoxy, —C(═O)—O-A, —C(═O)—N(A′)A, C(A′)(═N-OA), N(A′)A, N(A′)-C(═O)-A,
   A,A′ independently of one another are hydrogen, C₁-C₆-alkyl, C₂-C₆-alkenyl, C₂-C₆-alkynyl, phenyl, where the organic radicals may be partially or fully halogenated or may be substituted by C₁-C₄-alkoxy, or A and A′ together with the atoms to which they are attached are a five- or six-membered saturated heterocycle which comprises one or two heteroatoms from the group consisting of O, N and S;
      where the aliphatic groups of the radical definitions of L for their part may be partially or fully halogenated.

Particular preference is also given to 2-substituted pyrimidines of the formula I″

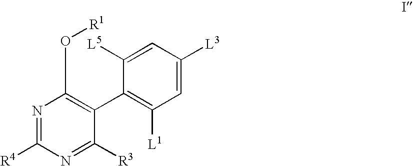

where
R¹ is C₁-C₆-alkyl, C₃-C₆-cycloalkyl, C₁-C₆-haloalkyl, C₃-C₆-halocycloalkyl, or (C₁-C₆-alkyl)C₃-C₆-cycloalkyl;
R³ is halogen, C₁-C₄-alkoxy or C₁-C₆-alkylthio;
R⁴ is pyrazole, 1,2,4-triazole, pyridinyl, cyano, C(═N—OCH₃)NH₂ or CONH₂;
L¹ is chlorine or fluorine;
L³ fluorine or C₁-C₄-alkoxy, preferably fluorine or methoxy;
L⁵ is hydrogen or fluorine.

Particular preference is also given to 2-substituted pyrimidines of the formula I″

where
R¹ is C₁-C₆-alkyl or C₁-C₆-haloalkyl;
R³ is halogen;
R⁴ is pyrazole, 1,2,4-triazole, C(═N—OCH₃)NH₂ or CONH₂
L¹ is chlorine or fluorine
L³ fluorine
L⁵ is hydrogen or fluorine,
   in particular where
R¹ is ethyl, propyl, isopropyl, 1,2-dimethylpropyl, 1,2,2-trimethylpropyl, 1-methyl-2,2,2-trifluoroethyl or 2,2,2-trifluoroethyl;
R³ is fluorine or chlorine;
R⁴ is pyrazole, 1,2,4-triazole, C(═N—OCH₃)NH₂ or CONH₂;
L¹ is chlorine or fluorine;
L³ is fluorine;
L⁵ is hydrogen or fluorine.

In particular with a view to their use, preference is given to compounds I compiled in the tables below. Moreover, the groups mentioned for a substituent in the tables are per se, independently of the combination in which they are mentioned, a particularly preferred embodiment of the substituent in question.

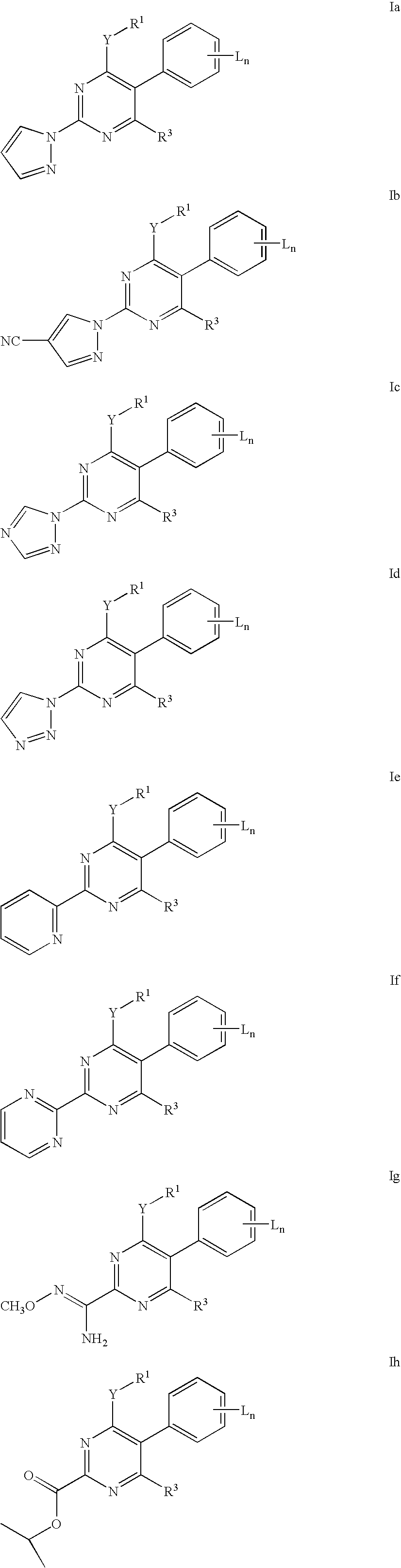

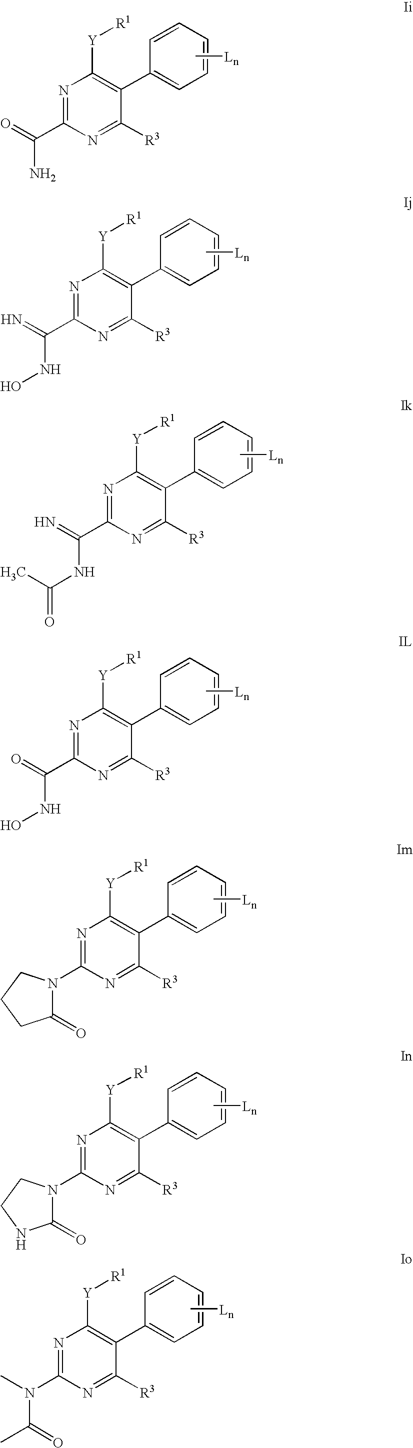

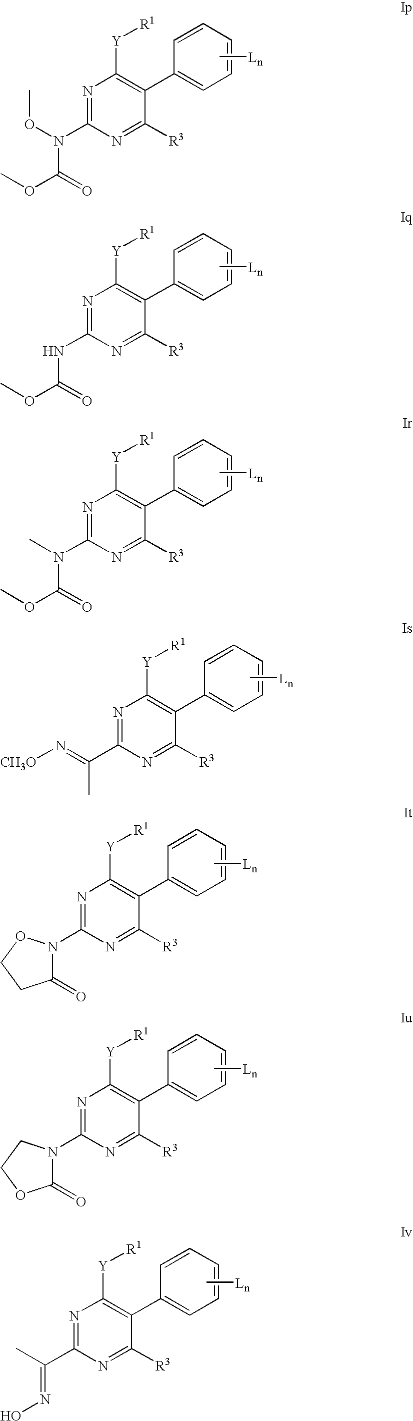

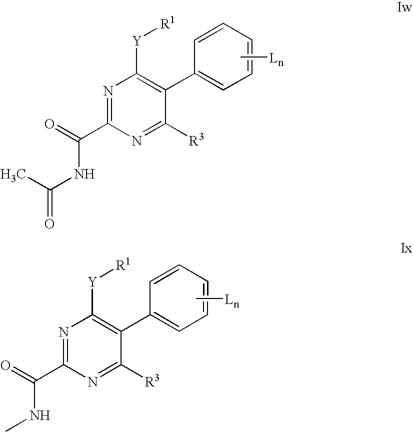

### Table 1

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-chloro, R³ is methyl and YR¹ for each compound corresponds to one row of Table A

### Table 2

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro, R³ is methyl and YR¹ for each compound corresponds to one row of Table A

### Table 3

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dichloro, R³ is methyl and YR¹ for each compound corresponds to one row of Table A

### Table 4

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-methyl, R³ is methyl and YR¹ for each compound corresponds to one row of Table A

### Table 5

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trifluoro, R³ is methyl and YR¹ for each compound corresponds to one row of Table A

### Table 6

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-fluoro, R³ is methyl and YR¹ for each compound corresponds to one row of Table A

### Table 7

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxycarbonyl, R³ is methyl and YR¹ for each compound corresponds to one row of Table A

### Table 8

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-CN, R³ is methyl and YR¹ for each compound corresponds to one row of Table A

### Table 9

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,5-trifluoro, R³ is methyl and YR¹ for each compound corresponds to one row of Table A

### Table 10

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dichloro, R³ is methyl and YR¹ for each compound corresponds to one row of Table A

### Table 11

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro, R³ is methyl and YR¹ for each compound corresponds to one row of Table A

### Table 12

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro, R³ is methyl and YR¹ for each compound corresponds to one row of Table A

### Table 13

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-difluoro, R³ is methyl and YR¹ for each compound corresponds to one row of Table A

### Table 14

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-chloro, R³ is methyl and YR¹ for each compound corresponds to one row of Table A

### Table 15

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro-4-fluoro, R³ is methyl and YR¹ for each compound corresponds to one row of Table A

### Table 16

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3-difluoro, R³ is methyl and YR¹ for each compound corresponds to one row of Table A

### Table 17

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-difluoro, R³ is methyl and YR¹ for each compound corresponds to one row of Table A

### Table 18

Compounds of the formulae. Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3,4-trifluoro, R³ is methyl and YR¹ for each compound corresponds to one row of Table A

### Table 19

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl, R³ is methyl and YR¹ for each compound corresponds to one row of Table A

### Table 20

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dimethyl, R³ is methyl and YR¹ for each compound corresponds to one row of Table A

### Table 21

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl-4-chloro, R³ is methyl and YR¹ for each compound corresponds to one row of Table A

### Table 22

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-methyl, R³ is methyl and YR¹ for each compound corresponds to one row of Table A

### Table 23

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dimethyl, R³ is methyl and YR¹ for each compound corresponds to one row of Table A

### Table 24

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trimethyl, R³ is methyl and YR¹ for each compound corresponds to one row of Table A

### Table 25

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-cyano, R³ is methyl and YR¹ for each compound corresponds to one row of Table A

### Table 26

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methyl, R³ is methyl and YR¹ for each compound corresponds to one row of Table A

### Table 27

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methoxycarbonyl, R³ is methyl and YR¹ for each compound corresponds to one row of Table A

### Table 28

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxy, R³ is methyl and YR¹ for each compound corresponds to one row of Table A

### Table 29

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methyl, R³ is methyl and YR¹ for each compound corresponds to one row of Table A

### Table 30

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxycarbonyl, R³ is methyl and YR¹ for each compound corresponds to one row of Table A

### Table 31

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-bromo, R³ is methyl and YR¹ for each compound corresponds to one row of Table A

### Table 32

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-cyano, R³ is methyl and YR¹ for each compound corresponds to one row of Table A

### Table 33

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro,4-methoxy, R³ is methyl and YR¹ for each compound corresponds to one row of Table A

### Table 34

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,3-methyl, R³ is methyl and YR¹ for each compound corresponds to one row of Table A

### Table 35

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl, R³ is methyl and YR¹ for each compound corresponds to one row of Table A

### Table 36

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-cyano, R³ is methyl and YR¹ for each compound corresponds to one row of Table A

### Table 37

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-bromo, R³ is methyl and YR¹ for each compound corresponds to one row of Table A

### Table 38

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,5-fluoro, R³ is methyl and YR¹ for each compound corresponds to one row of Table A

### Table 39

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxy, R³ is methyl and YR¹ for each compound corresponds to one row of Table A

### Table 40

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxycarbonyl, R³ is methyl and YR¹ for each compound corresponds to one row of Table A

### Table 41

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl,4-bromo, R³ Methyl and YR¹ for each compound corresponds to one row of Table A

### Table 42

Compounds of the formulae Ia, Ib, Ic, Id, Ie, If, Ig, Ih, ii, Ij, Ik, Il, Im, In, Io, Ip, Iq, Ir, Is, It, Iu, Iv, Iw and Ix in which Lₙ is 2-fluoro,4-bromo, R³ is methyl and YR¹ for each compound corresponds to one row of Table A

### Table 43

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxy, R¹ is methyl and YR¹ for each compound corresponds to one row of Table A

### Table 44

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,5-methyl, R³ is methyl and YR¹ for each compound corresponds to one row of Table A

### Table 45

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is pentafluoro, R³ is methyl and YR¹ for each compound corresponds to one row of Table A

### Table 46

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-chloro, R³ is chloro and YR¹ for each compound corresponds to one row of Table A

### Table 47

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro, R³ is chloro and YR¹ for each compound corresponds to one row of Table A

### Table 48

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dichloro, R³ is chloro and YR¹ for each compound corresponds to one row of Table A

### Table 49

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-methyl, R³ is chloro and YR¹ for each compound corresponds to one row of Table A

### Table 50

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trifluoro, R³ is chloro and YR¹ for each compound corresponds to one row of Table A

### Table 51

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-fluoro, R³ is chloro and YR¹ for each compound corresponds to one row of Table A

### Table 52

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxycarbonyl, R³ is chloro and YR¹ for each compound corresponds to one row of Table A

### Table 53

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-CN, R³ is chloro and YR¹ for each compound corresponds to one row of Table A

### Table 54

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,5-trifluoro, R³ is chloro and YR¹ for each compound corresponds to one row of Table A

### Table 55

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dichloro, R³ is chloro and YR¹ for each compound corresponds to one row of Table A

### Table 56

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro, R³ is chloro and YR¹ for each compound corresponds to one row of Table A

### Table 57

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro, R³ is chloro and YR¹ for each compound corresponds to one row of Table A

### Table 58

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-difluoro, R³ is chloro and YR¹ for each compound corresponds to one row of Table A

### Table 59

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-chloro, R³ is chloro and YR¹ for each compound corresponds to one row of Table A

### Table 60

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro-4-fluoro, R³ is chloro and YR¹ for each compound corresponds to one row of Table A

### Table 61

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3-difluoro, R³ is chloro and YR¹ for each compound corresponds to one row of Table A

### Table 62

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-difluoro, R³ is chloro and YR¹ for each compound corresponds to one row of Table A

### Table 63

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3,4-trifluoro, R³ is chloro and YR¹ for each compound corresponds to one row of Table A

### Table 64

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl, R³ is chloro and YR¹ for each compound corresponds to one row of Table A

### Table 65

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dimethyl, R³ is chloro and YR¹ for each compound corresponds to one row of Table A

### Table 66

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl-4-chloro, R³ is chloro and YR¹ for each compound corresponds to one row of Table A

### Table 67

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-methyl, R¹ is chloro and YR¹ for each compound corresponds to one row of Table A

### Table 68

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dimethyl, R³ is chloro and YR¹ for each compound corresponds to one row of Table A

### Table 69

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trimethyl, R³ is chloro and YR¹ for each compound corresponds to one row of Table A

### Table 70

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-cyano, R³ is chloro and YR¹ for each compound corresponds to one row of Table A

### Table 71

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methyl, R³ is chloro and YR¹ for each compound corresponds to one row of Table A

### Table 72

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methoxycarbonyl, R³ is chloro and YR¹ for each compound corresponds to one row of Table A

### Table 73

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxy, R³ is chloro and YR¹ for each compound corresponds to one row of Table A

### Table 74

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methyl, R³ is chloro and YR¹ for each compound corresponds to one row of Table A

### Table 75

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxycarbonyl, R³ is chloro and YR¹ for each compound corresponds to one row of Table A

### Table 76

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-bromo, R³ is chloro and YR¹ for each compound corresponds to one row of Table A

### Table 77

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-cyano, R³ is chloro and YR¹ for each compound corresponds to one row of Table A

### Table 78

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro,4-methoxy, R³ is chloro and YR¹ for each compound corresponds to one row of Table A

### Table 79

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,3-methyl, R³ is chloro and YR¹ for each compound corresponds to one row of Table A

### Table 80

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl, R³ is chloro and YR¹ for each compound corresponds to one row of Table A

### Table 81

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-cyano, R³ is chloro and YR¹ for each compound corresponds to one row of Table A

### Table 82

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-bromo, R³ is chloro and YR¹ for each compound corresponds to one row of Table A

### Table 83

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,5-fluoro, R³ is chloro and YR¹ for each compound corresponds to one row of Table A

### Table 84

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxy, R³ is chloro and YR¹ for each compound corresponds to one row of Table A

### Table 85

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxycarbonyl, R³ is chloro and YR¹ for each compound corresponds to one row of Table A

### Table 86

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl,4-bromo, R³ is chloro and YR¹ for each compound corresponds to one row of Table A

### Table 87

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-bromo, R³ is chloro and YR¹ for each compound corresponds to one row of Table A

### Table 88

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxy, R³ is chloro and YR¹ for each compound corresponds to one row of Table A

### Table 89

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,5-methyl, R³ is chloro and YR¹ for each compound corresponds to one row of Table A

### Table 90

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which L is pentafluoro, R³ is chloro and YR¹ for each compound corresponds to one row of Table A

### Table 91

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-chloro, R³ is methoxy and YR¹ for each compound corresponds to one row of Table A

### Table 92

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro, R³ is methoxy and YR¹ for each compound corresponds to one row of Table A

### Table 93

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dichloro, R³ is methoxy and YR¹ for each compound corresponds to one row of Table A

### Table 94

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-methyl, R³ is methoxy and YR¹ for each compound corresponds to one row of Table A

### Table 95

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trifluoro, R³ is methoxy and YR¹ for each compound corresponds to one row of Table A

### Table 96

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-fluoro, R³ is methoxy and YR¹ for each compound corresponds to one row of Table A

### Table 97

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxycarbonyl, R³ is methoxy and YR¹ for each compound corresponds to one row of Table A

### Table 98

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-CN, R³ is methoxy and YR¹ for each compound corresponds to one row of Table A

### Table 99

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,5-trifluoro, R³ is methoxy and YR¹ for each compound corresponds to one row of Table A

### Table 100

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dichloro, R³ is methoxy and YR¹ for each compound corresponds to one row of Table A

### Table 101

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro, R³ is methoxy and YR¹ for each compound corresponds to one row of Table A

### Table 102

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro, R³ is methoxy and YR¹ for each compound corresponds to one row of Table A

### Table 103

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-difluoro, R³ is methoxy and YR¹ for each compound corresponds to one row of Table A

### Table 104

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-chloro, R³ is methoxy and YR¹ for each compound corresponds to one row of Table A

### Table 105

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro-4-fluoro, R³ is methoxy and YR¹ for each compound corresponds to one row of Table A

### Table 106

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3-difluoro, R³ is methoxy and YR¹ for each compound corresponds to one row of Table A

### Table 107

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-difluoro, R³ is methoxy and YR¹ for each compound corresponds to one row of Table A

### Table 108

Compounds of the formulae Ia, Ib, Ic, Id, Ie, If, Ig, Ih, Ii, Ij, Il, Il, Im, In, Io, Ip, Iq, Ir, Is, It, Iu, Iv, Iw and Ix in which Lₙ is 2,3,4-trifluoro, R³ is methoxy and YR¹ for each compound corresponds to one row of Table A

### Table 109

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl, R³ is methoxy and YR¹ for each compound corresponds to one row of Table A

### Table 110

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dimethyl, R³ is methoxy and YR¹ for each compound corresponds to one row of Table A

### Table 111

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl-4-chloro, R³ is methoxy and YR¹ for each compound corresponds to one row of Table A

### Table 112

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-methyl, R³ is methoxy and YR¹ for each compound corresponds to one row of Table A

### Table 113

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dimethyl, R³ is methoxy and YR¹ for each compound corresponds to one row of Table A

### Table 114

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trimethyl, R³ is methoxy and YR¹ for each compound corresponds to one row of Table A

### Table 115

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-cyano, R³ is methoxy and YR¹ for each compound corresponds to one row of Table A

### Table 116

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methyl, R³ is methoxy and YR¹ for each compound corresponds to one row of Table A

### Table 117

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methoxycarbonyl, R³ is methoxy and YR¹ for each compound corresponds to one row of Table A

### Table 118

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxy, R³ is methoxy and YR¹ for each compound corresponds to one row of Table A

### Table 119

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methyl, R³ is methoxy and YR¹ for each compound corresponds to one row of Table A

### Table 120

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxycarbonyl, R³ is methoxy and YR¹ for each compound corresponds to one row of Table A

### Table 121

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxy, R³ is methoxy and YR¹ for each compound corresponds to one row of Table A

### Table 122

Compounds of the formulae Ia, Ib, Ic, Id, Ie, If, Ig, Ih, Ii, Ij, Ik, Il, Im, In, Io, Ip, Iq, Ir, Is, It, Iu, iv, Iw and Ix in which Lₙ is 2-chloro,4-cyano, R³ is methoxy and YR¹ for each compound corresponds to one row of Table A

### Table 123

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro,4-methoxy, R³ is methoxy and YR¹ for each compound corresponds to one row of Table A

### Table 124

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,3-methyl, R³ is methoxy and YR¹ for each compound corresponds to one row of Table A

### Table 125

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl, R³ is methoxy and YR¹ for each compound corresponds to one row of Table A

### Table 126

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-cyano, R³ is methoxy and YR¹ for each compound corresponds to one row of Table A

### Table 127

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-bromo, R³ is methoxy and YR¹ for each compound corresponds to one row of Table A

### Table 128

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,5-fluoro, R³ is methoxy and YR¹ for each compound corresponds to one row of Table A

### Table 129

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxy, R³ is methoxy and YR¹ for each compound corresponds to one row of Table A

### Table 130

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxycarbonyl, R³ is methoxy and YR¹, for each compound corresponds to one row of Table A

### Table 131

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl,4-bromo, R³ is methoxy and YR¹ for each compound corresponds to one row of Table A

### Table 132

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-bromo, R³ is methoxy and YR¹ for each compound corresponds to one row of Table A

### Table 133

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxy, R³ is methoxy and YR¹ for each compound corresponds to one row of Table A

### Table 134

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,5-methyl, R³ is methoxy and YR¹ for each compound corresponds to one row of Table A

### Table 135

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is pentafluoro, R³ is methoxy and YR¹ for each compound corresponds to one row of Table A

### Table 136

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-chloro, R³ is cyano and YR¹ for each compound corresponds to one row of Table A

### Table 137

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro, R³ is cyano and YR¹, for each compound corresponds to one row of Table A

### Table 138

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dichloro, R³ is cyano and YR¹ for each compound corresponds to one row of Table A

### Table 139

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,6-methyl, R³ Cyano and YR¹ for each compound corresponds to one row of Table A

### Table 140

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trifluoro, R³ is cyano and YR¹ for each compound corresponds to one row of Table A

### Table 141

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-fluoro, R³ is cyano and YR¹ for each compound corresponds to one row of Table A

### Table 142

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxycarbonyl, R³ is cyano and YR¹ for each compound corresponds to one row of Table A

### Table 143

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-CN, R³ is cyano and YR¹ for each compound corresponds to one row of Table A

### Table 144

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,5-trifluoro, R³ is cyano and YR¹ for each compound corresponds to one row of Table A

### Table 145

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dichloro, R³ is cyano and YR¹ for each compound corresponds to one row of Table A

### Table 146

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro, R³ is cyano and YR¹ for each compound corresponds to one row of Table A

### Table 147

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro, R³ is cyano and YR¹ for each compound corresponds to one row of Table A

### Table 148

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-difluoro, R³ is cyano and YR¹ for each compound corresponds to one row of Table A

### Table 149

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-chloro, R³ is cyano and YR¹ for each compound corresponds to one row of Table A

### Table 150

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro-4-fluoro, R³ is cyano and YR¹ for each compound corresponds to one row of Table A

### Table 151

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3-difluoro, R³ is cyano and YR¹ for each compound corresponds to one row of Table A

### Table 152

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-difluoro, R³ is cyano and YR¹ for each compound corresponds to one row of Table A

### Table 153

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,3,4-trifluoro, R³ is cyano and YR¹ for each compound corresponds to one row of Table A

### Table 154

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl, R³ is cyano and YR¹ for each compound corresponds to one row of Table A

### Table 155

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4-dimethyl, R³ is cyano and YR¹ for each compound corresponds to one row of Table A

### Table 156

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl-4-chloro, R³ is cyano and YR¹ for each compound corresponds to one row of Table A

### Table 157

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro-4-methyl, R³ is cyano and YR¹ for each compound corresponds to one row of Table A

### Table 158

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-dimethyl, R³ is cyano and YR¹ for each compound corresponds to one row of Table A

### Table 159

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,4,6-trimethyl, R³ is cyano and YR¹ for each compound corresponds to one row of Table A

### Table 160

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-cyano, R³ is cyano and YR¹ for each compound corresponds to one row of Table A

### Table 161

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methyl, R³ is cyano and YR¹ for each compound corresponds to one row of Table A

### Table 162

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro-4-methoxycarbonyl, R³ is cyano and YR¹ for each compound corresponds to one row of Table A

### Table 163

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methoxy, R³ is cyano and YR¹ for each compound corresponds to one row of Table A

### Table 164

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-methyl, R³ is cyano and YR¹ for each compound corresponds to one row of Table A

### Table 165

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-Chlor,4-methoxycarbonyl, R³ is cyano and YR¹ for each compound corresponds to one row of Table A

### Table 166

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-bromo, R³ is cyano and YR¹ for each compound corresponds to one row of Table A

### Table 167

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro,4-cyano, R³ is cyano and YR¹ for each compound corresponds to one row of Table A

### Table 168

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,6-difluoro,4-methoxy, R³ is cyano and YR¹ for each compound corresponds to one row of Table A

### Table 169

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,3-methyl, R³ is cyano and YR¹ for each compound corresponds to one row of Table A

### Table 170

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl, R³ is cyano and YR¹ for each compound corresponds to one row of Table A

### Table 171

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-cyano, R³ is cyano and YR¹ for each compound corresponds to one row of Table A

### Table 172

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-bromo, R³ is cyano and YR¹ for each compound corresponds to one row of Table A

### Table 173

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,5-fluoro, R³ is cyano and YR¹ for each compound corresponds to one row of Table A

### Table 174

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxy, R³ is cyano and YR¹ for each compound corresponds to one row of Table A

### Table 175

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-methyl,4-methoxycarbonyl, R³ is cyano and YR¹ for each compound corresponds to one row of Table A

### Table 176

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2,5-dimethyl,4-bromo, R³ is cyano and YR¹ for each compound corresponds to one row of Table A

### Table 177

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-bromo, R³ is cyano and YR¹ for each compound corresponds to one row of Table A

### Table 178

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,4-methoxy, R³ is cyano and YR¹ for each compound corresponds to one row of Table A

### Table 179

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-fluoro,5-methyl, R³ is cyano and YR¹ for each compound corresponds to one row of Table A

### Table 180

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is pentafluoro, R³ is cyano and YR¹ for each compound corresponds to one row of Table A

### Table 181

Compounds of the formulae Ia, Ib, Ic, Id, Ie, If, Ig, Ih, Ii, Ij, Ik, Il, Im, In, Io, Ip, Iq, Ir, Is, It, Iu, Iv, Iw and Ix in which Lₙ is 2-chloro-5-fluoro, R³ is chloro and YR¹ for each compound corresponds to one row of Table A

TABLE A

Y—R¹

| No. | R¹ | Y |
|---|---|---|
| A-1 | CH₂CH₃ | O |
| A-2 | CH₂CH₂CH₃ | O |
| A-3 | CH₂CH₂F | O |
| A-4 | CH₂CF₃ | O |
| A-5 | CH₂CCl₃ | O |
| A-6 | CH(CH₃)₂ | O |
| A-7 | CH₂C(CH₃)₃ | O |
| A-8 | CH₂CH(CH₃)₂ | O |
| A-9 | (±CH(CH₂CH₃)CH₃ | O |
| A-10 | (R)CH(CH₂CH₃)CH₃ | O |
| A-11 | (SCH(CH₂CH₃)CH₃ | O |
| A-12 | (±CH(CH₃)—CH(CH₃)₂ | O |
| A-13 | (RCH(CH₃)—CH(CH₃)₂ | O |
| A-14 | (SCH(CH₃)—CH(CH₃)₂ | O |
| A-15 | (±CH(CH₃)—C(CH₃)₃ | O |
| A-16 | (RCH(CH₃)—C(CH₃)₃ | O |
| A-17 | (SCH(CH₃)—C(CH₃)₃ | O |
| A-18 | (±CH(CH₃)—CF₃ | O |
| A-19 | (RCH(CH₃)—CF₃ | O |
| A-20 | (SCH(CH₃)—CF₃ | O |
| A-21 | (±CH(CH₃)—CCl₃ | O |
| A-22 | (RCH(CH₃)—CCl₃ | O |
| A-23 | (SCH(CH₃)—CCl₃ | O |
| A-24 | CH₂C(CH₃)═CH₂ | O |
| A-25 | cyclopentyl | O |
| A-26 | cyclohexyl | O |
| A-27 | (CH₂)₃CH₃ | O |
| A-28 | C(CH₃)₃ | O |
| A-29 | (CH₂)₄CH₃ | O |
| A-30 | CH(CH₂CH₃)₂ | O |
| A-31 | CH₂CH₂CH(CH₃)₂ | O |
| A-32 | (±CH(CH₃)(CH₂)₂CH₃ | O |
| A-33 | (RCH(CH₃)(CH₂)₂CH₃ | O |
| A-34 | (SCH(CH₃)(CH₂)₂CH₃ | O |
| A-35 | (±CH₂CH(CH₃)CH₂CH₃ | O |
| A-36 | (RCH₂CH(CH₃)CH₂CH₃ | O |
| A-37 | (SCH₂CH(CH₃)CH₂CH₃ | O |
| A-38 | (±CH(CH₃)CH(CH₃)₂ | O |
| A-39 | (RCH(CH₃)CH(CH₃)₂ | O |
| A-40 | (SCH(CH₃)CH(CH₃)₂ | O |
| A-41 | (CH₂)₅CH₃ | O |
| A-42 | (±,±CH(CH₃)CH(CH₃)CH₂CH₃ | O |
| A-43 | (±,RCH(CH₃)CH(CH₃)CH₂CH₃ | O |
| A-44 | (±,SCH(CH₃)CH(CH₃)CH₂CH₃ | O |
| A-45 | (±CH₂CH(CH₃)CF₃ | O |
| A-46 | (RCH₂CH(CH₃)CF₃ | O |
| A-47 | (SCH₂CH(CH₃)CF₃ | O |
| A-48 | (±CH₂CH(CF₃)CH₂CH₃ | O |
| A-49 | (RCH₂CH(CF₃)CH₂CH₃ | O |
| A-50 | (SCH₂CH(CF₃)CH₂CH₃ | O |
| A-51 | (±,±CH(CH₃)CH(CH₃)CF₃ | O |
| A-52 | (±,RCH(CH₃)CH(CH₃)CF₃ | O |
| A-53 | (±,SCH(CH₃)CH(CH₃)CF₃ | O |
| A-54 | (±,±CH(CH₃)CH(CF₃)CH₂CH₃ | O |
| A-55 | (±,RCH(CH₃)CH(CF₃)CH₂CH₃ | O |
| A-56 | (±,SCH(CH₃)CH(CF₃)CH₂CH₃ | O |
| A-57 | CF₃ | O |
| A-58 | CF₂CF₃ | O |
| A-59 | CF₂CF₂CF₃ | O |
| A-60 | c-C₃H₅ | O |
| A-61 | (1-CH₃)-c-C₃H₄ | O |
| A-62 | c-C₅H₉ | O |
| A-63 | c-C₆H₁₁ | O |
| A-64 | (4-CH₃)-c-C₆H₁₀ | O |
| A-65 | CH₂C(CH₃)═CH₂ | O |
| A-66 | CH₂CH₂C(CH₃)═CH₂ | O |
| A-67 | CH₂—C(CH₃)₃ | O |
| A-68 | CH₂—Si(CH₃)₃ | O |
| A-69 | n-C₆H₁₃ | O |
| A-70 | (CH₂)₃—CH(CH₃)₂ | O |
| A-71 | (CH₂)₂—CH(CH₃)—C₂H₅ | O |
| A-72 | CH₂—CH(CH₃)-n-C₃H₇ | O |
| A-73 | CH(CH₃)-n-C₄H₉ | O |
| A-74 | CH₂—CH(C₂H₅)₂ | O |
| A-75 | CH(C₂H₅)-n-C₃H₇ | O |
| A-76 | CH₂-c-C₅H₉ | O |
| A-77 | CH₂—CH(CH₃)—CH(CH₃)₂ | O |
| A-78 | CH(CH₃)—CH₂CH(CH₃)₂ | O |
| A-79 | CH(CH₃)—CH(CH₃)—C₂H₅ | O |
| A-80 | CH(CH₃)—C(CH₃)₃ | O |
| A-81 | (CH₂)₂—C(CH₃)₃ | O |
| A-82 | CH₂—C(CH₃)₂—C₂H₅ | O |
| A-83 | 2-CH₃-c-C₅H₈ | O |
| A-84 | 3-CH₃-c-C₅H₈ | O |
| A-85 | C(CH₃)₂-n-C₃H₇ | O |
| A-86 | (CH₂)₆—CH₃ | O |
| A-87 | (CH₂)₄—CH(CH₃)₂ | O |
| A-88 | (CH₂)₃—CH(CH₃)—C₂H₅ | O |
| A-89 | (CH₂)₂—CH(CH₃)-n-C₃H₇ | O |
| A-90 | CH₂—CH(CH₃)-n-C₄H₉ | O |
| A-91 | CH(CH₃)-n-C₅H₁₁ | O |
| A-92 | (CH₂)₃C(CH₃)₃ | O |
| A-93 | (CH₂)₂CH(CH₃)—CH(CH₃)₂ | O |
| A-94 | (CH₂)CH(CH₃)—CH₂CH(CH₃)₂ | O |
| A-95 | CH(CH₃)(CH₂)₂—CH(CH₃)₂ | O |
| A-96 | (CH₂)₂C(CH₃)₂C₂H₅ | O |
| A-97 | CH₂CH(CH₃)CH(CH₃)C₂H₅ | O |
| A-98 | CH(CH₃)CH₂CH(CH₃)C₂H₅ | O |
| A-99 | CH₂C(CH₃)₂-n-C₃H₇ | O |
| A-100 | CH(CH₃)CH(CH₃)-n-C₃H₇ | O |
| A-101 | C(CH₃)₂-n-C₄H₉ | O |
| A-102 | (CH₂)₂CH(C₂H₅)₂ | O |
| A-103 | CH₂CH(C₂H₅)-n-C₃H₇ | O |
| A-104 | CH(C₂H₅)-n-C₄H₉ | O |
| A-105 | CH₂CH(CH₃)C(CH₃)₃ | O |
| A-106 | CH(CH₃)CH₂C(CH₃)₃ | O |
| A-107 | CH₂C(CH₃)₂CH(CH₃)₂ | O |
| A-108 | CH₂CH(C₂H₅)CH(CH₃)₂ | O |
| A-109 | CH(CH₃)CH(CH₃)CH(CH₃)₂ | O |
| A-110 | C(CH₃)₂CH₂CH(CH₃)₂ | O |
| A-111 | CH(C₂H₅)CH₂CH(CH₃)₂ | O |
| A-112 | CH(CH₃)C(CH₃)₂C₂H₅ | O |
| A-113 | CH(CH₃)CH(C₂H₅)₂ | O |
| A-114 | C(CH₃)₂CH(CH₃)C₂H₅ | O |
| A-115 | CH(C₂H₅)CH(CH₃)C₂H₅ | O |
| A-116 | C(CH₃)(C₂H₅)-n-C₃H₇ | O |
| A-117 | CH(n-C₃H₇)₂ | O |
| A-118 | CH(n-C₃H₇)CH(CH₃)₂ | O |
| A-119 | C(CH₃)₂C(CH₃)₃ | O |
| A-120 | C(CH₃)(C₂H₅)—CH(CH₃)₂ | O |
| A-121 | C(C₂H₅)₃ | O |
| A-122 | (3-CH₃)-c-C₆H₁₀ | O |
| A-123 | (2-CH₃)-c-C₆H₁₀ | O |
| A-124 | n-C₈H₁₇ | O |
| A-125 | CH₂C(═NO—CH₃)CH₃ | O |
| A-126 | CH₂C(═NO—C₂H₅)CH₃ | O |
| A-127 | CH₂C(═NO-n-C₃H₇)CH₃ | O |
| A-128 | CH₂C(═NO-i-C₃H₇)CH₃ | O |
| A-129 | CH(CH₃)C(═NOCH₃)CH₃ | O |
| A-130 | CH(CH₃)C(═NOC₂H₅)CH₃ | O |
| A-131 | CH(CH₃)C(═NO-n-C₃H₇)CH₃ | O |
| A-132 | CH(CH₃)C(═NO-i-C₃H₇)CH₃ | O |
| A-133 | C(═NOCH₃)C(═NOCH₃)CH₃ | O |
| A-134 | C(═NOCH₃)C(═NOC₂H₅)CH₃ | O |
| A-135 | C(═NOCH₃)C(═NO-n-C₃H₇)CH₃ | O |
| A-136 | C(═NOCH₃)C(═NO-i-C₃H₇)CH₃ | O |
| A-137 | C(═NOC₂H₅)C(═NOCH₃)CH₃ | O |
| A-138 | C(═NOC₂H₅)C(═NOC₂H₅)CH₃ | O |
| A-139 | C(═NOC₂H₅)C(═NO-n-C₃H₇)CH₃ | O |
| A-140 | C(═NOC₂H₅)C(═NO-i-C₃H₇)CH₃ | O |
| A-141 | CH₂C(═NO—CH₃)C₂H₅ | O |
| A-142 | CH₂C(═NO—C₂H₅)C₂H₅ | O |
| A-143 | CH₂C(═NO-n-C₃H₇)C₂H₅ | O |
| A-144 | CH₂C(═NO-i-C₃H₇)C₂H₅ | O |
| A-145 | CH(CH₃)C(═NOCH₃)C₂H₅ | O |
| A-146 | CH(CH₃)C(═NOC₂H₅)C₂H₅ | O |
| A-147 | CH(CH₃)C(═NO-n-C₃H₇)C₂H₅ | O |
| A-148 | CH(CH₃)C(═NO-n-C₃H₇)C₂H₅ | O |
| A-149 | C(═NOCH₃)C(═NOCH₃)C₂H₅ | O |
| A-150 | C(═NOCH₃)C(═NOC₂H₅)C₂H₅ | O |
| A-151 | C(═NOCH₃)C(═NO-n-C₃H₇)C₂H₅ | O |
| A-152 | C(═NOCH₃)C(═NO-i-C₃H₇)C₂H₅ | O |
| A-153 | C(═NOC₂H₅)C(═NOCH₃)C₂H₅ | O |
| A-154 | C(═NOC₂H₅)C(═NOC₂H₅)C₂H₅ | O |
| A-155 | C(═NOC₂H₅)C(═NO-n-C₃H₇)C₂H₅ | O |
| A-156 | C(═NOC₂H₅)C(═NO-i-C₃H₇)C₂H₅ | O |
| A-157 | CH═CH—CH₂CH₃ | O |
| A-158 | CH₂—CH═CH—CH₃ | O |
| A-159 | CH₂—CH₂—CH═CH₂ | O |
| A-160 | C(CH₃)₂CH₂CH₃ | O |
| A-161 | CH═C(CH₃)₂ | O |
| A-162 | C(═CH₂)—CH₂CH₃ | O |
| A-163 | C(CH₃)═CH—CH₃ | O |
| A-164 | CH(CH₃)CH═CH₂ | O |
| A-165 | CH═CH-n-C₃H₇ | O |
| A-166 | CH₂—CH═CH—C₂H₅ | O |
| A-167 | (CH₂)₂—CH═CH—CH₃ | O |
| A-168 | (CH₂)₃—CH═CH₂ | O |
| A-169 | CH═CH—CH(CH₃)₂ | O |
| A-170 | CH₂—CH═C(CH₃)₂ | O |
| A-171 | (CH₂)₂—C(CH₃)═CH₂ | O |
| A-172 | CH═C(CH₃)—C₂H₅ | O |
| A-173 | CH₂—C(═CH₂)—C₂H₅ | O |
| A-174 | CH₂—C(CH₃)═CH—CH₃ | O |
| A-175 | CH₂—CH(CH₃)—CH═CH₂ | O |
| A-176 | C(═CH₂)—CH₂—CH₂—CH₃ | O |
| A-177 | C(CH₃)═CH—CH₂—CH₃ | O |
| A-178 | CH(CH₃)—CH═CH—CH₃ | O |
| A-179 | CH(CH₃)—CH₂—CH═CH₂ | O |
| A-180 | C(═CH₂)CH(CH₃)₂ | O |
| A-181 | C(CH₃)═C(CH₃)₂ | O |
| A-182 | CH(CH₃)—C(═CH₂)—CH₃ | O |
| A-183 | C(CH₃)₂—CH═CH₂ | O |
| A-184 | C(C₂H₅)═CH—CH₃ | O |
| A-185 | CH(C₂H₅)—CH═CH₂ | O |
| A-186 | CH═CH—CH₂—CH₂—CH₂—CH₃ | O |
| A-187 | CH₂—CH═CH—CH₂—CH₂—CH₃ | O |
| A-188 | CH₂—CH₂—CH═CH—CH₂—CH₃ | O |
| A-189 | CH₂—CH₂—CH₂—CH═CH—CH₃ | O |
| A-190 | CH₂—CH₂—CH₂—CH₂—CH═CH₂ | O |
| A-191 | CH═CH—CH₂—CH(CH₃)CH₃ | O |
| A-192 | CH₂—CH═CH—CH(CH₃)CH₃ | O |
| A-193 | CH₂—CH₂—CH═C(CH₃)CH₃ | O |
| A-194 | CH₂—CH₂—CH₂—C(CH₃)═CH₂ | O |
| A-195 | CH═CH—CH(CH₃)—CH₂—CH₃ | O |
| A-196 | CH₂—CH═C(CH₃)—CH₂—CH₃ | O |
| A-197 | CH₂—CH₂—C(═CH₂)—CH₂—CH₃ | O |
| A-198 | CH₂—CH₂—C(CH₃)═CH—CH₃ | O |
| A-199 | CH₂—CH₂—CH(CH₃)—CH═CH₂ | O |
| A-200 | CH═C(CH₃)—CH₂—CH₂—CH₃ | O |
| A-201 | CH₂—C(═CH₂)—CH₂—CH₂—CH₃ | O |
| A-202 | CH₂—C(CH₃)═CH—CH₂—CH₃ | O |
| A-203 | CH₂—CH(CH₃)—CH═CH—CH₃ | O |
| A-204 | CH₂—CH(CH₃)—CH₂—CH═CH₂ | O |
| A-205 | C(═CH₂)—CH₂—CH₂—CH₂—CH₃ | O |
| A-206 | C(CH₃)═CH—CH₂—CH₂—CH₃ | O |
| A-207 | CH(CH₃)—CH═CH—CH₂—CH₃ | O |
| A-208 | CH(CH₃)—CH₂—CH═CH—CH₃ | O |
| A-209 | CH(CH₃)—CH₂—CH₂—CH═CH₂ | O |
| A-210 | CH═CH—C(CH₃)₃ | O |
| A-211 | CH═C(CH₃)—CH(CH₃)—CH₃ | O |
| A-212 | CH₂—C(═CH₂)—CH(CH₃)—CH₃ | O |
| A-213 | CH₂—C(CH₃)═C(CH₃)—CH₃ | O |
| A-214 | CH₂—CH(CH₃)—C(═CH₂)—CH₃ | O |
| A-215 | C(═CH₂)—CH₂—CH(CH₃)—CH₃ | O |
| A-216 | C(CH₃)═CH—CH(CH₃)—CH₃ | O |
| A-217 | CH(CH₃)—CH═C(CH₃)—CH₃ | O |
| A-218 | CH(CH₃)—CH₂—C(═CH₂)—CH₃ | O |
| A-219 | CH═C(CH₂—CH₃)—CH₂—CH₃ | O |
| A-220 | CH₂—C(═CH—CH₃)—CH₂—CH₃ | O |
| A-221 | CH₂—CH(CH═CH₂)—CH₂—CH₃ | O |
| A-222 | C(═CH—CH₃)—CH₂—CH₂—CH₃ | O |
| A-223 | CH(CH═CH₂)—CH₂—CH₂—CH₃ | O |
| A-224 | C(CH₂—CH₃)═CH—CH₂—CH₃ | O |
| A-225 | CH(CH₂—CH₃)—CH═CH—CH₃ | O |
| A-226 | CH(CH₂—CH₃)—CH₂—CH═CH₂ | O |
| A-227 | CH₂—C(CH₃)₂—CH═CH₂ | O |
| A-228 | C(═CH₂)—CH(CH₃)—CH₂—CH₃ | O |
| A-229 | C(CH₃)═C(CH₃)—CH₂—CH₃ | O |
| A-230 | CH(CH₃)—C(═CH₂)—CH₂—CH₃ | O |
| A-231 | CH(CH₃)—C(CH₃)═CH—CH₃ | O |
| A-232 | CH(CH₃)—CH(CH₃)—CH═CH₂ | O |
| A-233 | C(CH₃)₂—CH═CH—CH₃ | O |
| A-234 | C(CH₃)₂—CH₂—CH═CH₂ | O |
| A-235 | C(═CH₂)—C(CH₃)₃ | O |
| A-236 | C(═CH—CH₃)—CH(CH₃)—CH₃ | O |
| A-237 | CH(CH═CH₂)—CH(CH₃)—CH₃ | O |
| A-238 | C(CH₂—CH₃)═C(CH₃)—CH₃ | O |
| A-239 | CH(CH₂—CH₃)—C(═CH₂)—CH₃ | O |
| A-240 | C(CH₃)₂—C(═CH₂)—CH₃ | O |
| A-241 | C(CH₃)(CH═CH₂)—CH₂—CH₃ | O |
| A-242 | C(CH₃)(CH₂CH₃)—CH₂—CH₂—CH₃ | O |
| A-243 | CH(CH₂CH₃)—CH(CH₃)—CH₂—CH₃ | O |
| A-244 | CH(CH₂CH₃)—CH₂—CH(CH₃)—CH₃ | O |
| A-245 | C(CH₃)₂—C(CH₃)₃ | O |
| A-246 | C(CH₂—CH₃)—C(CH₃)₃ | O |
| A-247 | C(CH₃)(CH₂—CH₃)—CH(CH₃)₂ | O |
| A-248 | CH(CH(CH₃)₂)—CH(CH₃)₂ | O |
| A-249 | CH═CH—CH₂—CH₂—CH₂—CH₂—CH₃ | O |
| A-250 | CH₂—CH═CH—CH₂—CH₂—CH₂—CH₃ | O |
| A-251 | CH₂—CH₂—CH═CH—CH₂—CH₂—CH₃ | O |
| A-252 | CH₂—CH₂—CH₂—CH═CH—CH₂—CH₃ | O |
| A-253 | CH₂—CH₂—CH₂—CH₂—CH═CH—CH₃ | O |
| A-254 | CH₂—CH₂—CH₂—CH₂—CH₂—CH═CH₂ | O |
| A-255 | CH═CH—CH₂—CH₂—CH(CH₃)—CH₃ | O |
| A-256 | CH₂—CH═CH—CH₂—CH(CH₃)—CH₃ | O |
| A-257 | CH₂—CH₂—CH═CH—CH(CH₃)—CH₃ | O |
| A-258 | CH₂—CH₂—CH₂—CH═C(CH₃)—CH₃ | O |
| A-259 | CH₂—CH₂—CH₂—CH₂—C(═CH₂)—CH₃ | O |
| A-260 | CH═CH—CH₂—CH(CH₃)—CH₂—CH₃ | O |
| A-261 | CH₂—CH═CH—CH(CH₃)—CH₂—CH₃ | O |
| A-262 | CH₂—CH₂—CH═C(CH₃)—CH₂—CH₃ | O |
| A-263 | CH₂—CH₂—CH₂—C(═CH₂)—CH₂—CH₃ | O |
| A-264 | CH₂—CH₂—CH₂—C(CH₃)═CH—CH₃ | O |
| A-265 | CH₂—CH₂—CH₂—CH(CH₃)—CH═CH₂ | O |
| A-266 | CH═CH—CH(CH₃)—CH₂—CH₂—CH₃ | O |
| A-267 | CH₂—CH═C(CH₃)—CH₂—CH₂—CH₃ | O |
| A-268 | CH₂—CH₂—C(═CH₂)—CH₂—CH₂—CH₃ | O |
| A-269 | CH₂—CH₂—C(CH₃)═CH—CH₂—CH₃ | O |
| A-270 | CH₂—CH₂—CH(CH₃)—CH═CH—CH₃ | O |
| A-271 | CH₂—CH₂—CH(CH₃)—CH₂—CH═CH₂ | O |
| A-272 | CH═C(CH₃)—CH₂—CH₂—CH₂—CH₃ | O |
| A-273 | CH₂—C(═CH₂)—CH₂—CH₂—CH₂—CH₃ | O |
| A-274 | CH₂—C(CH₃)═CH—CH₂—CH₂—CH₃ | O |
| A-275 | CH₂—CH(CH₃)—CH═CH—CH₂—CH₃ | O |
| A-276 | CH₂—CH(CH₃)—CH₂—CH═CH—CH₃ | O |
| A-277 | CH₂—CH(CH₃)—CH₂—CH₂—CH═CH₂ | O |
| A-278 | C(═CH₂)—CH₂—CH₂—CH₂—CH₂—CH₃ | O |
| A-279 | C(CH₃)═CH—CH₂—CH₂—CH₂—CH₃ | O |
| A-280 | CH(CH₃)—CH═CH—CH₂—CH₂—CH₃ | O |
| A-281 | CH(CH₃)—CH₂—CH═CH—CH₂—CH₃ | O |
| A-282 | CH(CH₃)—CH₂—CH₂—CH═CH—CH₃ | O |
| A-283 | CH(CH₃)—CH₂—CH₂—CH₂—CH═CH₂ | O |
| A-284 | CH═CH—CH₂—C(CH₃)₃ | O |
| A-285 | CH₂—CH═CH—C(CH₃)₃ | O |
| A-286 | CH═CH—CH(CH₃)—CH(CH₃)₂ | O |
| A-287 | CH₂—CH═C(CH₃)—CH(CH₃)₂ | O |
| A-288 | CH₂—CH₂—C(═CH₂)—CH(CH₃)₂ | O |
| A-289 | CH₂—CH₂—C(CH₃)═C(CH₃)₂ | O |
| A-290 | CH₂—CH₂—CH(CH₃)—C(═CH₂)—CH₃ | O |
| A-291 | CH═C(CH₃)—CH₂—CH(CH₃)₂ | O |
| A-292 | CH₂—C(═CH₂)—CH₂—CH(CH₃)₂ | O |
| A-293 | CH₂—C(CH₃)═CH—CH(CH₃)₂ | O |
| A-294 | CH₂—CH(CH₃)—CH═C(CH₃)₂ | O |
| A-295 | CH₂—CH(CH₃)—CH₂—C(═CH₂)—CH₃ | O |
| A-296 | C(═CH₂)—CH₂—CH₂—CH(CH₃)₂ | O |
| A-297 | C(CH₃)═CH—CH₂—CH(CH₃)₂ | O |
| A-298 | CH(CH₃)—CH═CH—CH(CH₃)₂ | O |
| A-299 | CH(CH₃)—CH₂—CH═C(CH₃)₂ | O |
| A-300 | CH(CH₃)—CH₂—CH₂—C(═CH₂)—CH₃ | O |
| A-301 | CH═CH—C(CH₃)₂—CH₂—CH₃ | O |
| A-302 | CH₂—CH₂—C(CH₃)₂—CH═CH₂ | O |
| A-303 | CH═C(CH₃)—CH(CH₃)—CH₂—CH₃ | O |
| A-304 | CH₂—C(═CH₂)—CH(CH₃)—CH₂—CH₃ | O |
| A-305 | CH₂—C(CH₃)═C(CH₃)—CH₂—CH₃ | O |
| A-306 | CH₂—CH(CH₃)—C(═CH₂)—CH₂—CH₃ | O |
| A-307 | CH₂—CH(CH₃)—C(CH₃)═CH—CH₃ | O |
| A-308 | CH₂—CH(CH₃)—CH(CH₃)—CH═CH₂ | O |
| A-309 | C(═CH₂)—CH₂—CH(CH₃)—CH₂—CH₃ | O |
| A-310 | C(CH₃)═CH—CH(CH₃)—CH₂—CH₃ | O |
| A-311 | CH(CH₃)—CH═C(CH₃)—CH₂—CH₃ | O |
| A-312 | CH(CH₃)—CH₂—C(═CH₂)—CH₂—CH₃ | O |
| A-313 | CH(CH₃)—CH₂—C(CH₃)═CH—CH₃ | O |
| A-314 | CH(CH₃)—CH₂—CH(CH₃)—CH═CH₂ | O |
| A-315 | CH₂—C(CH₃)₂—CH═CH—CH₃ | O |
| A-316 | CH₂—C(CH₃)₂—CH₂—CH═CH₂ | O |
| A-317 | C(═CH₂)—CH(CH₃)—CH₂—CH₂—CH₃ | O |
| A-318 | C(CH₃)═C(CH₃)—CH₂—CH₂—CH₃ | O |
| A-319 | CH(CH₃)—C(═CH₂)—CH₂—CH₂—CH₃ | O |
| A-320 | CH(CH₃)—C(CH₃)═CH—CH₂—CH₃ | O |
| A-321 | CH(CH₃)—CH(CH₃)—CH═CH—CH₃ | O |
| A-322 | CH(CH₃)—CH(CH₃)—CH₂—CH═CH₂ | O |
| A-323 | C(CH₃)₂—CH═CH—CH₂—CH₃ | O |
| A-324 | C(CH₃)₂—CH₂—CH═CH—CH₃ | O |
| A-325 | C(CH₃)₂—CH₂—CH₂—CH═CH₂ | O |
| A-326 | CH═CH—CH(CH₂—CH₃)—CH₂—CH₃ | O |
| A-327 | CH₂—CH═C(CH₂—CH₃)—CH₂—CH₃ | O |
| A-328 | CH₂—CH₂—C(═CH—CH₃)—CH₂—CH₃ | O |
| A-329 | CH₂—CH₂—CH(CH═CH₂)—CH₂—CH₃ | O |
| A-330 | CH═C(CH₂—CH₃)—CH₂—CH₂—CH₃ | O |
| A-331 | CH₂—C(═CH—CH₃)—CH₂—CH₂—CH₃ | O |
| A-332 | CH₂—CH(CH═CH₂)—CH₂—CH₂—CH₃ | O |
| A-333 | CH₂—C(CH₂—CH₃)═CH—CH₂—CH₃ | O |
| A-334 | CH₂—CH(CH₂—CH₃)—CH═CH—CH₃ | O |
| A-335 | CH₂—CH(CH₂—CH₃)—CH—CH═CH₂ | O |
| A-336 | C(═CH—CH₃)—CH₂—CH₂—CH₂—CH₃ | O |
| A-337 | CH(CH═CH₂)—CH₂—CH₂—CH₂—CH₃ | O |
| A-338 | C(CH₂—CH₃)═CH—CH₂—CH₂—CH₃ | O |
| A-339 | CH(CH₂—CH₃)—CH═CH—CH₂—CH₃ | O |
| A-340 | CH(CH₂—CH₃)—CH₂—CH═CH—CH₃ | O |
| A-341 | CH(CH₂—CH₃)—CH₂—CH₂—CH═CH₂ | O |
| A-342 | C(═CH—CH₂—CH₃)—CH₂—CH₂—CH₃ | O |
| A-343 | C(CH═CH—CH₃)—CH₂—CH₂—CH₃ | O |
| A-344 | C(CH₂—CH═CH₂)—CH₂—CH₂—CH₃ | O |
| A-345 | CH═C(CH₃)—C(CH₃)₃ | O |
| A-346 | CH₂—C(═CH₂)—C(CH₃)₃ | O |
| A-347 | CH₂—C(CH₃)₂—CH(═CH₂)—CH₃ | O |
| A-348 | C(═CH₂)—CH(CH₃)—CH(CH₃)—CH₃ | O |
| A-349 | C(CH₃)═C(CH₃)—CH(CH₃)—CH₃ | O |
| A-350 | CH(CH₃)—C(═CH₂)—CH(CH₃)—CH₃ | O |
| A-351 | CH(CH₃)—C(CH₃)═C(CH₃)—CH₃ | O |
| A-352 | CH(CH₃)—CH(CH₃)—C(═CH₂)—CH₃ | O |
| A-353 | C(CH₃)₂—CH═C(CH₃)—CH₃ | O |
| A-354 | C(CH₃)₂—CH₂—C(═CH₂)—CH₃ | O |
| A-355 | C(CH₃)₂—C(═CH₂)—CH₂—CH₃ | O |
| A-356 | C(CH₃)₂—C(CH₃)═CH—CH₃ | O |
| A-357 | C(CH₃)₂—CH(CH₃)CH═CH₂ | O |
| A-358 | CH(CH₂—CH₃)—CH₂—CH(CH₃)—CH₃ | O |
| A-359 | CH(CH₂—CH₃)—CH(CH₃)—CH₂—CH₃ | O |
| A-360 | C(CH₃)(CH₂—CH₃)—CH₂—CH₂—CH₃ | O |
| A-361 | CH(i-C₃H₇)—CH₂—CH₂—CH₃ | O |
| A-362 | CH═C(CH₂—CH₃)—CH(CH₃)—CH₃ | O |
| A-363 | CH₂—C(═CH—CH₃)—CH(CH₃)—CH₃ | O |
| A-364 | CH₂—CH(CH═CH₂)—CH(CH₃)—CH₃ | O |
| A-365 | CH₂—C(CH₂—CH₃)═C(CH₃)—CH₃ | O |
| A-366 | CH₂—CH(CH₂—CH₃)—C(═CH₂)—CH₃ | O |
| A-367 | CH₂—C(CH₃)(CH═CH₂)—CH₂—CH₃ | O |
| A-368 | C(═CH₂)—CH(CH₂—CH₃)—CH₂—CH₃ | O |
| A-369 | C(CH₃)═C(CH₂—CH₃)—CH₂—CH₃ | O |
| A-370 | CH(CH₃)—C(═CH—CH₃)—CH₂—CH₃ | O |
| A-371 | CH(CH₃)—CH(CH═CH₂)—CH₂—CH₃ | O |
| A-372 | CH═C(CH₂—CH₃)—CH(CH₃)—CH₃ | O |
| A-373 | CH₂—C(═CH—CH₃)—CH(CH₃)—CH₃ | O |
| A-374 | CH₂—CH(CH═CH₂)—CH(CH₃)—CH₃ | O |
| A-375 | CH₂—C(CH₂—CH₃)═C(CH₃)—CH₃ | O |
| A-376 | CH₂—CH(CH₂—CH₃)—C(═CH₂)—CH₃ | O |
| A-377 | C(═CH—CH₃)—CH₂—CH(CH₃)—CH₃ | O |
| A-378 | CH(CH═CH₂)—CH₂—CH(CH₃)—CH₃ | O |
| A-379 | C(CH₂—CH₃)═CH—CH(CH₃)—CH₃ | O |
| A-380 | CH(CH₂—CH₃)CH═C(CH₃)—CH₃ | O |
| A-381 | CH(CH₂—CH₃)CH₂—C(═CH₂)—CH₃ | O |
| A-382 | C(═CH—CH₃)CH(CH₃)—CH₂—CH₃ | O |
| A-383 | CH(CH═CH₂)CH(CH₃)—CH₂—CH₃ | O |
| A-384 | C(CH₂—CH₃)═C(CH₃)—CH₂—CH₃ | O |
| A-385 | CH(CH₂—CH₃)—C(═CH₂)—CH₂—CH₃ | O |
| A-386 | CH(CH₂—CH₃)—C(CH₃)═CH—CH₃ | O |
| A-387 | CH(CH₂—CH₃)—CH(CH₃)—CH═CH₂ | O |
| A-388 | C(CH₃)(CH═CH₂)—CH₂—CH₂—CH₃ | O |
| A-389 | C(CH₃)(CH₂—CH₃)—CH═CH—CH₃ | O |
| A-390 | C(CH₃)(CH₂—CH₃)—CH₂—CH═CH₂ | O |
| A-391 | C[═C(CH₃)—CH₃]—CH₂—CH₂—CH₃ | O |
| A-392 | CH[C(═CH₂)—CH₃]—CH₂—CH₂—CH₃ | O |
| A-393 | C(i-C₃H₇)═CH—CH₂—CH₃ | O |
| A-394 | CH(i-C₃H₇)—CH═CH—CH₃ | O |
| A-395 | CH(i-C₃H₇)—CH₂—CH═CH₂ | O |
| A-396 | C(═CH—CH₃)—C(CH₃)₃ | O |
| A-397 | CH(CH═CH₂)—C(CH₃)₃ | O |
| A-398 | C(CH₃)(CH═CH₂)CH(CH₃)—CH₃ | O |
| A-399 | C(CH₃)(CH₂—CH₃)C(═CH₂)—CH₃ | O |
| A-400 | 2-CH₃-cyclohex-1-enyl | O |
| A-401 | [2-(═CH₂)]-c-C₆H₉ | O |
| A-402 | 2-CH₃-cyclohex-2-enyl | O |
| A-403 | 2-CH₃-cyclohex-3-enyl | O |
| A-404 | 2-CH₃-cyclohex-4-enyl | O |
| A-405 | 2-CH₃-cyclohex-5-enyl | O |
| A-406 | 2-CH₃-cyclohex-6-enyl | O |
| A-407 | 3-CH₃-cyclohex-1-enyl | O |
| A-408 | 3-CH₃-cyclohex-2-enyl | O |
| A-409 | [3-(═CH₂)]-c-C₆H₉ | O |
| A-410 | 3-CH₃-cyclohex-3-enyl | O |
| A-411 | 3-CH₃-cyclohex-4-enyl | O |
| A-412 | 3-CH₃-cyclohex-5-enyl | O |
| A-413 | 3-CH₃-cyclohex-6-enyl | O |
| A-414 | 4-CH₃-cyclohex-1-enyl | O |
| A-415 | 4-CH₃-cyclohex-2-enyl | O |
| A-416 | 4-CH₃-cyclohex-3-enyl | O |
| A-417 | [4-(═CH₂)]-c-C₆H₉ | O |
| A-418 | CH₂CH₃ | S |
| A-419 | CH₂CH₂CH₃ | S |
| A-420 | CH₂CH₂F | S |
| A-421 | CH₂CF₃ | S |
| A-422 | CH₂CCl₃ | S |
| A-423 | CH(CH₃)₂ | S |
| A-424 | CH₂C(CH₃)₃ | S |
| A-425 | CH₂CH(CH₃)₂ | S |
| A-426 | (±CH(CH₂CH₃)CH₃ | S |
| A-427 | (R)CH(CH₂CH₃)CH₃ | S |
| A-428 | (S)CH(CH₂CH₃)CH₃ | S |
| A-429 | (±)CH(CH₃)—CH(CH₃)₂ | S |
| A-430 | (R)CH(CH₃)—CH(CH₃)₂ | S |
| A-431 | (S)CH(CH₃)—CH(CH₃)₂ | S |
| A-432 | (±)CH(CH₃)—C(CH₃)₃ | S |
| A-433 | (R)CH(CH₃)—C(CH₃)₃ | S |
| A-434 | (S)CH(CH₃)—C(CH₃)₃ | S |
| A-435 | (±)CH(CH₃)—CF₃ | S |
| A-436 | (R)CH(CH₃)—CF₃ | S |
| A-437 | (S)CH(CH₃)—CF₃ | S |
| A-438 | (±)CH(CH₃)—CCl₃ | S |
| A-439 | (R)CH(CH₃)—CCl₃ | S |
| A-440 | (S)CH(CH₃)—CCl₃ | S |
| A-441 | CH₂C(CH₃)═CH₂ | S |
| A-442 | Cyclopentyl | S |
| A-443 | Cyclohexyl | S |
| A-444 | (CH₂)₃CH₃ | S |
| A-445 | C(CH₃)₃ | S |
| A-446 | (CH₂)₄CH₃ | S |
| A-447 | CH(CH₂CH₃)₂ | S |
| A-448 | CH₂CH₂CH(CH₃)₂ | S |
| A-449 | (±CH(CH₃)(CH₂)₂CH₃ | S |
| A-450 | (RCH(CH₃)(CH₂)₂CH₃ | S |
| A-451 | (SCH(CH₃)(CH₂)₂CH₃ | S |
| A-452 | (±CH₂CH(CH₃)CH₂CH₃ | S |
| A-453 | (RCH₂CH(CH₃)CH₂CH₃ | S |
| A-454 | (SCH₂CH(CH₃)CH₂CH₃ | S |
| A-455 | (±CH(CH₃)CH(CH₃)₂ | S |
| A-456 | (RCH(CH₃)CH(CH₃)₂ | S |
| A-457 | (SCH(CH₃)CH(CH₃)₂ | S |
| A-458 | (CH₂)₅CH₃ | S |
| A-459 | (±,±CH(CH₃)CH(CH₃)CH₂CH₃ | S |
| A-460 | (±,RCH(CH₃)CH(CH₃)CH₂CH₃ | S |
| A-461 | (±,SCH(CH₃)CH(CH₃)CH₂CH₃ | S |
| A-462 | (±CH₂CH(CH₃)CF₃ | S |
| A-463 | (RCH₂CH(CH₃)CF₃ | S |
| A-464 | (SCH₂CH(CH₃)CF₃ | S |
| A-465 | (±CH₂CH(CF₃)CH₂CH₃ | S |
| A-466 | (RCH₂CH(CF₃)CH₂CH₃ | S |
| A-467 | (SCH₂CH(CF₃)CH₂CH₃ | S |
| A-468 | (±,±CH(CH₃)CH(CH₃)CF₃ | S |
| A-469 | (±,RCH(CH₃)CH(CH₃)CF₃ | S |
| A-470 | (±,SCH(CH₃)CH(CH₃)CF₃ | S |
| A-471 | (±,±CH(CH₃)CH(CF₃)CH₂CH₃ | S |
| A-472 | (±,RCH(CH₃)CH(CF₃)CH₂CH₃ | S |
| A-473 | (±,SCH(CH₃)CH(CF₃)CH₂CH₃ | S |
| A-474 | CF₃ | S |
| A-475 | CF₂CF₃ | S |
| A-476 | CF₂CF₂CF₃ | S |
| A-477 | c-C₃H₅ | S |
| A-478 | (1-CH₃)-c-C₃H₄ | S |
| A-479 | c-C₅H₉ | S |
| A-480 | c-C₆H₁₁ | S |
| A-481 | (4-CH₃)-c-C₆H₁₀ | S |
| A-482 | CH₂C(CH₃)═CH₂ | S |
| A-483 | CH₂CH₂C(CH₃)═CH₂ | S |
| A-484 | CH₂—C(CH₃)₃ | S |
| A-485 | CH₂—Si(CH₃)₃ | S |
| A-486 | n-C₆H₁₃ | S |
| A-487 | (CH₂)₃—CH(CH₃)₂ | S |
| A-488 | (CH₂)₂—CH(CH₃)—C₂H₅ | S |
| A-489 | CH₂—CH(CH₃)-n-C₃H₇ | S |
| A-490 | CH(CH₃)-n-C₄H₉ | S |
| A-491 | CH₂—CH(C₂H₅)₂ | S |
| A-492 | CH(C₂H₅)-n-C₃H₇ | S |
| A-493 | CH₂-c-C₅H₉ | S |
| A-494 | CH₂—CH(CH₃)—CH(CH₃)₂ | S |
| A-495 | CH(CH₃)—CH₂CH(CH₃)₂ | S |
| A-496 | CH(CH₃)—CH(CH₃)—C₂H₅ | S |
| A-497 | CH(CH₃)—C(CH₃)₃ | S |
| A-498 | (CH₂)₂—C(CH₃)₃ | S |
| A-499 | CH₂—C(CH₃)₂—C₂H₅ | S |
| A-500 | 2-CH₃-c-C₅H₈ | S |
| A-501 | 3-CH₃-c-C₅H₈ | S |
| A-502 | C(CH₃)₂-n-C₃H₇ | S |
| A-503 | (CH₂)₆—CH₃ | S |
| A-504 | (CH₂)₄—CH(CH₃)₂ | S |
| A-505 | (CH₂)₃—CH(CH₃)—C₂H₅ | S |
| A-506 | (CH₂)₂—CH(CH₃)-n-C₃H₇ | S |
| A-507 | CH₂—CH(CH₃)-n-C₄H₉ | S |
| A-508 | CH(CH₃)-n-C₅H₁₁ | S |
| A-509 | (CH₂)₃C(CH₃)₃ | S |
| A-510 | (CH₂)₂CH(CH₃)—CH(CH₃)₂ | S |
| A-511 | (CH₂)CH(CH₃)—CH₂CH(CH₃)₂ | S |
| A-512 | CH(CH₃)(CH₂)₂—CH(CH₃)₂ | S |
| A-513 | (CH₂)₂C(CH₃)₂C₂H₅ | S |
| A-514 | CH₂CH(CH₃)CH(CH₃)C₂H₅ | S |
| A-515 | CH(CH₃)CH₂CH(CH₃)C₂H₅ | S |
| A-516 | CH₂C(CH₃)₂-n-C₃H₇ | S |
| A-517 | CH(CH₃)CH(CH₃)-n-C₃H₇ | S |
| A-518 | C(CH₃)₂-n-C₄H₉ | S |
| A-519 | (CH₂)₂CH(C₂H₅)₂ | S |
| A-520 | CH₂CH(C₂H₅)-n-C₃H₇ | S |
| A-521 | CH(C₂H₅)-n-C₄H₉ | S |
| A-522 | CH₂CH(CH₃)C(CH₃)₃ | S |
| A-523 | CH(CH₃)CH₂C(CH₃)₃ | S |
| A-524 | CH₂C(CH₃)₂CH(CH₃)₂ | S |
| A-525 | CH₂CH(C₂H₅)CH(CH₃)₂ | S |
| A-526 | CH(CH₃)CH(CH₃)CH(CH₃)₂ | S |
| A-527 | C(CH₃)₂CH₂CH(CH₃)₂ | S |
| A-528 | CH(C₂H₅)CH₂CH(CH₃)₂ | S |
| A-529 | CH(CH₃)C(CH₃)₂C₂H₅ | S |
| A-530 | CH(CH₃)CH(C₂H₅)₂ | S |
| A-531 | C(CH₃)₂CH(CH₃)C₂H₅ | S |
| A-532 | CH(C₂H₅)CH(CH₃)C₂H₅ | S |
| A-533 | C(CH₃)(C₂H₅)-n-C₃H₇ | S |
| A-534 | CH(n-C₃H₇)₂ | S |
| A-535 | CH(n-C₃H₇)CH(CH₃)₂ | S |
| A-536 | C(CH₃)₂C(CH₃)₃ | S |
| A-537 | C(CH₃)(C₂H₅)—CH(CH₃)₂ | S |
| A-538 | C(C₂H₅)₃ | S |
| A-539 | (3-CH₃)-c-C₆H₁₀ | S |
| A-540 | (2-CH₃)-c-C₆H₁₀ | S |
| A-541 | n-C₈H₁₇ | S |
| A-542 | CH₂C(═NO—CH₃)CH₃ | S |
| A-543 | CH₂C(═NO—C₂H₅)CH₃ | S |
| A-544 | CH₂C(═NO-n-C₃H₇)CH₃ | S |
| A-545 | CH₂C(═NO-i-C₃H₇)CH₃ | S |
| A-546 | CH(CH₃)C(═NOCH₃)CH₃ | S |
| A-547 | CH(CH₃)C(═NOC₂H₅)CH₃ | S |
| A-548 | CH(CH₃)C(═NO-n-C₃H₇)CH₃ | S |
| A-549 | CH(CH₃)C(═NO-i-C₃H₇)CH₃ | S |
| A-550 | C(═NOCH₃)C(═NOCH₃)CH₃ | S |
| A-551 | C(═NOCH₃)C(═NOC₂H₅)CH₃ | S |
| A-552 | C(═NOCH₃)C(═NO-n-C₃H₇)CH₃ | S |
| A-553 | C(═NOCH₃)C(═NO-i-C₃H₇)CH₃ | S |
| A-554 | C(═NOC₂H₅)C(═NOCH₃)CH₃ | S |
| A-555 | C(═NOC₂H₅)C(═NOC₂H₅)CH₃ | S |
| A-556 | C(═NOC₂H₅)C(═NO-n-C₃H₇)CH₃ | S |
| A-557 | C(═NOC₂H₅)C(═NO-i-C₃H₇)CH₃ | S |
| A-558 | CH₂C(═NO—CH₃)C₂H₅ | S |
| A-559 | CH₂C(═NO—C₂H₅)C₂H₅ | S |
| A-560 | CH₂C(═NO-n-C₃H₇)C₂H₅ | S |
| A-561 | CH₂C(═NO-i-C₃H₇)C₂H₅ | S |
| A-562 | CH(CH₃)C(═NOCH₃)C₂H₅ | S |
| A-563 | CH(CH₃)C(═NOC₂H₅)C₂H₅ | S |
| A-564 | CH(CH₃)C(═NO-n-C₃H₇)C₂H₅ | S |
| A-565 | CH(CH₃)C(═NO-n-C₃H₇)C₂H₅ | S |
| A-566 | C(═NOCH₃)C(═NOCH₃)C₂H₅ | S |
| A-567 | C(═NOCH₃)C(═NOC₂H₅)C₂H₅ | S |
| A-568 | C(═NOCH₃)C(═NO-n-C₃H₇)C₂H₅ | S |
| A-569 | C(═NOCH₃)C(═NO-i-C₃H₇)C₂H₅ | S |
| A-570 | C(═NOC₂H₅)C(═NOCH₃)C₂H₅ | S |
| A-571 | C(═NOC₂H₅)C(═NOC₂H₅)C₂H₅ | S |
| A-572 | C(═NOC₂H₅)C(═NO-n-C₃H₇)C₂H₅ | S |
| A-573 | C(═NOC₂H₅)C(═NO-i-C₃H₇)C₂H₅ | S |
| A-574 | CH═CH—CH₂CH₃ | S |
| A-575 | CH₂—CH═CH—CH₃ | S |
| A-576 | CH₂—CH₂—CH═CH₂ | S |
| A-577 | C(CH₃)₂CH₂CH₃ | S |
| A-578 | CH═C(CH₃)₂ | S |
| A-579 | C(═CH₂)—CH₂CH₃ | S |
| A-580 | C(CH₃)═CH—CH₃ | S |
| A-581 | CH(CH₃)CH═CH₂ | S |
| A-582 | CH═CH-n-C₃H₇ | S |
| A-583 | CH₂—CH═CH—C₂H₅ | S |
| A-584 | (CH₂)₂—CH═CH—CH₃ | S |
| A-585 | (CH₂)₃—CH═CH₂ | S |
| A-586 | CH═CH—CH(CH₃)₂ | S |
| A-587 | CH₂—CH═C(CH₃)₂ | S |
| A-588 | (CH₂)₂—C(CH₃)═CH₂ | S |
| A-589 | CH═C(CH₃)—C₂H₅ | S |
| A-590 | CH₂—C(═CH₂)—C₂H₅ | S |
| A-591 | CH₂—C(CH₃)═CH—CH₃ | S |
| A-592 | CH₂—CH(CH₃)—CH═CH₂ | S |
| A-593 | C(═CH₂)—CH₂—CH₂—CH₃ | S |
| A-594 | C(CH₃)═CH—CH₂—CH₃ | S |
| A-595 | CH(CH₃)—CH═CH—CH₃ | S |
| A-596 | CH(CH₃)—CH₂—CH═CH₂ | S |
| A-597 | C(═CH₂)CH(CH₃)₂ | S |
| A-598 | C(CH₃)═C(CH₃)₂ | S |
| A-599 | CH(CH₃)—C(═CH₂)—CH₃ | S |
| A-600 | C(CH₃)₂—CH═CH₂ | S |
| A-601 | C(C₂H₅)═CH—CH₃ | S |
| A-602 | CH(C₂H₅)—CH═CH₂ | S |
| A-603 | CH═CH—CH₂—CH₂—CH₂—CH₃ | S |
| A-604 | CH₂—CH═CH—CH₂—CH₂—CH₃ | S |
| A-605 | CH₂—CH₂—CH═CH—CH₂—CH₃ | S |
| A-606 | CH₂—CH₂—CH₂—CH═CH—CH₃ | S |
| A-607 | CH₂—CH₂—CH₂—CH₂—CH═CH₂ | S |
| A-608 | CH═CH—CH₂—CH(CH₃)CH₃ | S |
| A-609 | CH₂—CH═CH—CH(CH₃)CH₃ | S |
| A-610 | CH₂—CH₂—CH═C(CH₃)CH₃ | S |
| A-611 | CH₂—CH₂—CH₂—C(CH₃)═CH₂ | S |
| A-612 | CH═CH—CH(CH₃)—CH₂—CH₃ | S |
| A-613 | CH₂—CH═C(CH₃)—CH₂—CH₃ | S |
| A-614 | CH₂—CH₂—C(═CH₂)—CH₂—CH₃ | S |
| A-615 | CH₂—CH₂—C(CH₃)═CH—CH₃ | S |
| A-616 | CH₂—CH₂—CH(CH₃)—CH═CH₂ | S |
| A-617 | CH═C(CH₃)—CH₂—CH₂—CH₃ | S |
| A-618 | CH₂—C(═CH₂)—CH₂—CH₂—CH₃ | S |
| A-619 | CH₂—C(CH₃)═CH—CH₂—CH₃ | S |
| A-620 | CH₂—CH(CH₃)—CH═CH—CH₃ | S |
| A-621 | CH₂—CH(CH₃)—CH₂—CH═CH₂ | S |
| A-622 | C(═CH₂)—CH₂—CH₂—CH₂—CH₃ | S |
| A-623 | C(CH₃)═CH—CH₂—CH₂—CH₃ | |
| A-624 | CH(CH₃)—CH═CH—CH₂—CH₃ | S |
| A-625 | CH(CH₃)—CH₂—CH═CH—CH₃ | S |
| A-626 | CH(CH₃)—CH₂—CH₂—CH═CH₂ | S |
| A-627 | CH═CH—C(CH₃)₃ | S |
| A-628 | CH═C(CH₃)—CH(CH₃)—CH₃ | S |
| A-629 | CH₂—C(═CH₂)—CH(CH₃)—CH₃ | S |
| A-630 | CH₂—C(CH₃)═C(CH₃)—CH₃ | S |
| A-631 | CH₂—CH(CH₃)—C(═CH₂)—CH₃ | S |
| A-632 | C(═CH₂)—CH₂—CH(CH₃)—CH₃ | S |
| A-633 | C(CH₃)═CH—CH(CH₃)—CH₃ | S |
| A-634 | CH(CH₃)—CH═C(CH₃)—CH₃ | S |
| A-635 | CH(CH₃)—CH₂—C(═CH₂)—CH₃ | S |
| A-636 | CH═C(CH₂—CH₃)—CH₂—CH₃ | S |
| A-637 | CH₂—C(═CH—CH₃)—CH₂—CH₃ | S |
| A-638 | CH₂—CH(CH═CH₂)—CH₂—CH₃ | S |
| A-639 | C(═CH—CH₃)—CH₂—CH₂—CH₃ | S |
| A-640 | CH(CH═CH₂)—CH₂—CH₂—CH₃ | S |
| A-641 | C(CH₂—CH₃)═CH—CH₂—CH₃ | S |
| A-642 | CH(CH₂—CH₃)—CH═CH—CH₃ | S |
| A-643 | CH(CH₂—CH₃)—CH₂—CH═CH₂ | S |
| A-644 | CH₂—C(CH₃)₂—CH═CH₂ | S |
| A-645 | C(═CH₂)—CH(CH₃)—CH₂—CH₃ | S |
| A-646 | C(CH₃)═C(CH₃)—CH₂—CH₃ | S |
| A-647 | CH(CH₃)—C(═CH₂)—CH₂—CH₃ | S |
| A-648 | CH(CH₃)—C(CH₃)═CH—CH₃ | S |
| A-649 | CH(CH₃)—CH(CH₃)—CH═CH₂ | S |
| A-650 | C(CH₃)₂—CH═CH—CH₃ | S |
| A-651 | C(CH₃)₂—CH₂—CH═CH₂ | S |
| A-652 | C(═CH₂)—C(CH₃)₃ | S |
| A-653 | C(═CH—CH₃)—CH(CH₃)—CH₃ | S |
| A-654 | CH(CH═CH₂)—CH(CH₃)—CH₃ | S |
| A-655 | C(CH₂—CH₃)═C(CH₃)—CH₃ | S |
| A-656 | CH(CH₂—CH₃)—C(═CH₂)—CH₃ | S |
| A-657 | C(CH₃)₂—C(═CH₂)—CH₃ | S |
| A-658 | C(CH₃)(CH═CH₂)—CH₂—CH₃ | S |
| A-659 | C(CH₃)(CH₂CH₃)—CH₂—CH₂—CH₃ | S |
| A-660 | CH(CH₂CH₃)—CH(CH₃)—CH₂—CH₃ | S |
| A-661 | CH(CH₂CH₃)—CH₂—CH(CH₃)—CH₃ | S |
| A-662 | C(CH₃)₂—C(CH₃)₃ | S |
| A-663 | C(CH₂—CH₃)—C(CH₃)₃ | S |
| A-664 | C(CH₃)(CH₂—CH₃)—CH(CH₃)₂ | S |
| A-665 | CH(CH(CH₃)₂)—CH(CH₃)₂ | S |
| A-666 | CH═CH—CH₂—CH₂—CH₂—CH₂—CH₃ | S |
| A-667 | CH₂—CH═CH—CH₂—CH₂—CH₂—CH₃ | S |
| A-668 | CH₂—CH₂—CH═CH—CH₂—CH₂—CH₃ | S |
| A-669 | CH₂—CH₂—CH₂—CH═CH—CH₂—CH₃ | S |
| A-670 | CH₂—CH₂—CH₂—CH₂—CH═CH—CH₃ | S |
| A-671 | CH₂—CH₂—CH₂—CH₂—CH₂—CH═CH₂ | S |
| A-672 | CH═CH—CH₂—CH₂—CH(CH₃)—CH₃ | S |
| A-673 | CH₂—CH═CH—CH₂—CH(CH₃)—CH₃ | S |
| A-674 | CH₂—CH₂—CH═CH—CH(CH₃)—CH₃ | S |
| A-675 | CH₂—CH₂—CH₂—CH═C(CH₃)—CH₃ | S |
| A-676 | CH₂—CH₂—CH₂—CH₂—C(═CH₂)—CH₃ | S |
| A-677 | CH═CH—CH₂—CH(CH₃)—CH₂—CH₃ | S |
| A-678 | CH₂—CH═CH—CH(CH₃)—CH₂—CH₃ | S |
| A-679 | CH₂—CH₂—CH═C(CH₃)—CH₂—CH₃ | S |
| A-680 | CH₂—CH₂—CH₂—C(═CH₂)—CH₂—CH₃ | S |
| A-681 | CH₂—CH₂—CH₂—C(CH₃)═CH—CH₃ | S |
| A-682 | CH₂—CH₂—CH₂—CH(CH₃)—CH═CH₂ | S |
| A-683 | CH═CH—CH(CH₃)—CH₂—CH₂—CH₃ | S |
| A-684 | CH₂—CH═C(CH₃)—CH₂—CH₂—CH₃ | S |
| A-685 | CH₂—CH₂—C(═CH₂)—CH₂—CH₂—CH₃ | S |
| A-686 | CH₂—CH₂—C(CH₃)═CH—CH₂—CH₃ | S |
| A-687 | CH₂—CH₂—CH(CH₃)—CH═CH—CH₃ | S |
| A-688 | CH₂—CH₂—CH(CH₃)—CH₂—CH═CH₂ | S |
| A-689 | CH═C(CH₃)—CH₂—CH₂—CH₂—CH₃ | S |
| A-690 | CH₂—C(═CH₂)—CH₂—CH₂—CH₂—CH₃ | S |
| A-691 | CH₂—C(CH₃)═CH—CH₂—CH₂—CH₃ | S |
| A-692 | CH₂—CH(CH₃)—CH═CH—CH₂—CH₃ | S |
| A-693 | CH₂—CH(CH₃)—CH₂—CH═CH—CH₃ | S |
| A-694 | CH₂—CH(CH₃)—CH₂—CH₂—CH═CH₂ | S |
| A-695 | C(═CH₂)—CH₂—CH₂—CH₂—CH₂—CH₃ | S |
| A-696 | C(CH₃)═CH—CH₂—CH₂—CH₂—CH₃ | S |
| A-697 | CH(CH₃)—CH═CH—CH₂—CH₂—CH₃ | S |
| A-698 | CH(CH₃)—CH₂—CH═CH—CH₂—CH₃ | S |
| A-699 | CH(CH₃)—CH₂—CH₂—CH═CH—CH₃ | S |
| A-700 | CH(CH₃)—CH₂—CH₂—CH₂—CH═CH₂ | S |
| A-701 | CH═CH—CH₂—C(CH₃)₃ | S |
| A-702 | CH₂—CH═CH—C(CH₃)₃ | S |
| A-703 | CH═CH—CH(CH₃)—CH(CH₃)₂ | S |
| A-704 | CH₂—CH═C(CH₃)—CH(CH₃)₂ | S |
| A-705 | CH₂—CH₂—C(═CH₂)—CH(CH₃)₂ | S |
| A-706 | CH₂—CH₂—C(CH₃)═C(CH₃)₂ | S |
| A-707 | CH₂—CH₂—CH(CH₃)—C(═CH₂)—CH₃ | S |
| A-708 | CH═C(CH₃)—CH₂—CH(CH₃)₂ | S |
| A-709 | CH₂—C(═CH₂)—CH₂—CH(CH₃)₂ | S |
| A-710 | CH₂—C(CH₃)═CH—CH(CH₃)₂ | S |
| A-711 | CH₂—CH(CH₃)—CH═C(CH₃)₂ | S |
| A-712 | CH₂—CH(CH₃)—CH₂—C(═CH₂)—CH₃ | S |
| A-713 | C(═CH₂)—CH₂—CH₂—CH(CH₃)₂ | S |
| A-714 | C(CH₃)═CH—CH₂—CH(CH₃)₂ | S |
| A-715 | CH(CH₃)—CH═CH—CH(CH₃)₂ | S |
| A-716 | CH(CH₃)—CH₂—CH═C(CH₃)₂ | S |
| A-717 | CH(CH₃)—CH₂—CH₂—C(═CH₂)—CH₃ | S |
| A-718 | CH═CH—C(CH₃)₂—CH₂—CH₃ | S |
| A-719 | CH₂—CH₂—C(CH₃)₂—CH═CH₂ | S |
| A-720 | CH═C(CH₃)—CH(CH₃)—CH₂—CH₃ | S |
| A-721 | CH₂—C(═CH₂)—CH(CH₃)—CH₂—CH₃ | S |
| A-722 | CH₂—C(CH₃)═C(CH₃)—CH₂—CH₃ | S |
| A-723 | CH₂—CH(CH₃)—C(═CH₂)—CH₂—CH₃ | S |
| A-724 | CH₂—CH(CH₃)—C(CH₃)═CH—CH₃ | S |
| A-725 | CH₂—CH(CH₃)—CH(CH₃)—CH═CH₂ | S |
| A-726 | C(═CH₂)—CH₂—CH(CH₃)—CH₂—CH₃ | S |
| A-727 | C(CH₃)═CH—CH(CH₃)—CH₂—CH₃ | S |
| A-728 | CH(CH₃)—CH═C(CH₃)—CH₂—CH₃ | S |
| A-729 | CH(CH₃)—CH₂—C(═CH₂)—CH₂—CH₃ | S |
| A-730 | CH(CH₃)—CH₂—C(CH₃)═CH—CH₃ | S |
| A-731 | CH(CH₃)—CH₂—CH(CH₃)—CH═CH₂ | S |
| A-732 | CH₂—C(CH₃)₂—CH═CH—CH₃ | S |
| A-733 | CH₂—C(CH₃)₂—CH₂—CH═CH₂ | S |
| A-734 | C(═CH₂)—CH(CH₃)—CH₂—CH₂—CH₃ | S |
| A-735 | C(CH₃)═C(CH₃)—CH₂—CH₂—CH₃ | S |
| A-736 | CH(CH₃)—C(═CH₂)—CH₂—CH₂—CH₃ | S |
| A-737 | CH(CH₃)—C(CH₃)═CH—CH₂—CH₃ | S |
| A-738 | CH(CH₃)—CH(CH₃)—CH═CH—CH₃ | S |
| A-739 | CH(CH₃)—CH(CH₃)—CH₂—CH═CH₂ | S |
| A-740 | C(CH₃)₂—CH═CH—CH₂—CH₃ | S |
| A-741 | C(CH₃)₂—CH₂—CH═CH—CH₃ | S |
| A-742 | C(CH₃)₂—CH₂—CH₂—CH═CH₂ | S |
| A-743 | CH═CH—CH(CH₂—CH₃)—CH₂—CH₃ | S |
| A-744 | CH₂—CH═C(CH₂—CH₃)—CH₂—CH₃ | S |
| A-745 | CH₂—CH₂—C(═CH—CH₃)—CH₂—CH₃ | S |
| A-746 | CH₂—CH₂—CH(CH═CH₂)—CH₂—CH₃ | S |
| A-747 | CH═C(CH₂—CH₃)—CH₂—CH₂—CH₃ | S |
| A-748 | CH₂—C(═CH—CH₃)—CH₂—CH₂—CH₃ | S |
| A-749 | CH₂—CH(CH═CH₂)—CH₂—CH₂—CH₃ | S |
| A-750 | CH₂—C(CH₂—CH₃)═CH—CH₂—CH₃ | S |
| A-751 | CH₂—CH(CH₂—CH₃)—CH═CH—CH₃ | S |
| A-752 | CH₂—CH(CH₂—CH₃)—CH—CH═CH₂ | S |
| A-753 | C(═CH—CH₃)—CH₂—CH₂—CH₂—CH₃ | S |
| A-754 | CH(CH═CH₂)—CH₂—CH₂—CH₂—CH₃ | S |
| A-755 | C(CH₂—CH₃)═CH—CH₂—CH₂—CH₃ | S |
| A-756 | CH(CH₂—CH₃)—CH═CH—CH₂—CH₃ | S |
| A-757 | CH(CH₂—CH₃)—CH₂—CH═CH—CH₃ | S |
| A-758 | CH(CH₂—CH₃)—CH₂—CH₂—CH═CH₂ | S |
| A-759 | C(═CH—CH₂—CH₃)—CH₂—CH₂—CH₃ | S |
| A-760 | C(CH═CH—CH₃)—CH₂—CH₂—CH₃ | S |
| A-761 | C(CH₂—CH═CH₂)—CH₂—CH₂—CH₃ | S |
| A-762 | CH═C(CH₃)—C(CH₃)₃ | S |
| A-763 | CH₂—C(═CH₂)—C(CH₃)₃ | S |
| A-764 | CH₂—C(CH₃)₂—CH(═CH₂)—CH₃ | S |
| A-765 | C(═CH₂)—CH(CH₃)—CH(CH₃)—CH₃ | S |
| A-766 | C(CH₃)═C(CH₃)—CH(CH₃)—CH₃ | S |
| A-767 | CH(CH₃)—C(═CH₂)—CH(CH₃)—CH₃ | S |
| A-768 | CH(CH₃)—C(CH₃)═C(CH₃)—CH₃ | S |
| A-769 | CH(CH₃)—CH(CH₃)—C(═CH₂)—CH₃ | S |
| A-770 | C(CH₃)₂—CH═C(CH₃)—CH₃ | S |
| A-771 | C(CH₃)₂—CH₂—C(═CH₂)—CH₃ | S |
| A-772 | C(CH₃)₂—C(═CH₂)—CH₂—CH₃ | S |
| A-773 | C(CH₃)₂—C(CH₃)═CH—CH₃ | S |
| A-774 | C(CH₃)₂—CH(CH₃)CH═CH₂ | S |
| A-775 | CH(CH₂—CH₃)—CH₂—CH(CH₃)—CH₃ | S |
| A-776 | CH(CH₂—CH₃)—CH(CH₃)—CH₂—CH₃ | S |
| A-777 | C(CH₃)(CH₂—CH₃)—CH₂—CH₂—CH₃ | S |
| A-778 | CH(i-C₃H₇)—CH₂—CH₂—CH₃ | S |
| A-779 | CH═C(CH₂—CH₃)—CH(CH₃)—CH₃ | S |
| A-780 | CH₂—C(═CH—CH₃)—CH(CH₃)—CH₃ | S |
| A-781 | CH₂—CH(CH═CH₂)—CH(CH₃)—CH₃ | S |
| A-782 | CH₂—C(CH₂—CH₃)═C(CH₃)—CH₃ | S |
| A-783 | CH₂—CH(CH₂—CH₃)—C(═CH₂)—CH₃ | S |
| A-784 | CH₂—C(CH₃)(CH═CH₂)—CH₂—CH₃ | S |
| A-785 | C(═CH₂)—CH(CH₂—CH₃)—CH₂—CH₃ | S |
| A-786 | C(CH₃)═C(CH₂—CH₃)—CH₂—CH₃ | S |
| A-787 | CH(CH₃)—C(═CH—CH₃)—CH₂—CH₃ | S |
| A-788 | CH(CH₃)—CH(CH═CH₂)—CH₂—CH₃ | S |
| A-789 | CH═C(CH₂—CH₃)—CH(CH₃)—CH₃ | S |
| A-790 | CH₂—C(═CH—CH₃)—CH(CH₃)—CH₃ | S |
| A-791 | CH₂—CH(CH═CH₂)—CH(CH₃)—CH₃ | S |
| A-792 | CH₂—C(CH₂—CH₃)═C(CH₃)—CH₃ | S |
| A-793 | CH₂—CH(CH₂—CH₃)—C(═CH₂)—CH₃ | S |
| A-794 | C(═CH—CH₃)—CH₂—CH(CH₃)—CH₃ | S |
| A-795 | CH(CH═CH₂)—CH₂—CH(CH₃)—CH₃ | S |
| A-796 | C(CH₂—CH₃)═CH—CH(CH₃)—CH₃ | S |
| A-797 | CH(CH₂—CH₃)CH═C(CH₃)—CH₃ | S |
| A-798 | CH(CH₂—CH₃)CH₂—C(═CH₂)—CH₃ | S |
| A-799 | C(═CH—CH₃)CH(CH₃)—CH₂—CH₃ | S |
| A-800 | CH(CH═CH₂)CH(CH₃)—CH₂—CH₃ | S |
| A-801 | C(CH₂—CH₃)═C(CH₃)—CH₂—CH₃ | S |
| A-802 | CH(CH₂—CH₃)—C(═CH₂)—CH₂—CH₃ | S |
| A-803 | CH(CH₂—CH₃)—C(CH₃)═CH—CH₃ | S |
| A-804 | CH(CH₂—CH₃)—CH(CH₃)—CH═CH₂ | S |
| A-805 | C(CH₃)(CH═CH₂)—CH₂—CH₂—CH₃ | S |
| A-806 | C(CH₃)(CH₂—CH₃)—CH═CH—CH₃ | S |
| A-807 | C(CH₃)(CH₂—CH₃)—CH₂—CH═CH₂ | S |
| A-808 | C[═C(CH₃)—CH₃]—CH₂—CH₂—CH₃ | S |
| A-809 | CH[C(═CH₂)—CH₃]—CH₂—CH₂—CH₃ | S |
| A-810 | C(i-C₃H₇)═CH—CH₂—CH₃ | S |
| A-811 | CH(i-C₃H₇)—CH═CH—CH₃ | S |
| A-812 | CH(i-C₃H₇)—CH₂—CH═CH₂ | S |
| A-813 | C(═CH—CH₃)—C(CH₃)₃ | S |
| A-814 | CH(CH═CH₂)—C(CH₃)₃ | S |
| A-815 | C(CH₃)(CH═CH₂)CH(CH₃)—CH₃ | S |
| A-816 | C(CH₃)(CH₂—CH₃)C(═CH₂)—CH₃ | S |
| A-817 | 2-CH₃-cyclohex-1-enyl | S |
| A-818 | [2-(═CH₂)]-c-C₆H₉ | S |
| A-819 | 2-CH₃-cyclohex-2-enyl | S |
| A-820 | 2-CH₃-cyclohex-3-enyl | S |
| A-821 | 2-CH₃-cyclohex-4-enyl | S |
| A-822 | 2-CH₃-cyclohex-5-enyl | S |
| A-823 | 2-CH₃-cyclohex-6-enyl | S |
| A-824 | 3-CH₃-cyclohex-1-enyl | S |
| A-825 | 3-CH₃-cyclohex-2-enyl | S |
| A-826 | [3-(═CH₂)]-c-C₆H₉ | S |
| A-827 | 3-CH₃-cyclohex-3-enyl | S |
| A-828 | 3-CH₃-cyclohex-4-enyl | S |
| A-829 | 3-CH₃-cyclohex-5-enyl | S |
| A-830 | 3-CH₃-cyclohex-6-enyl | S |
| A-831 | 4-CH₃-cyclohex-1-enyl | S |
| A-832 | 4-CH₃-cyclohex-2-enyl | S |
| A-833 | 4-CH₃-cyclohex-3-enyl | S |
| A-834 | [4-(═CH₂)]-c-C₆H₉ | S |

The compounds I are suitable as fungicides. They are distinguished through an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:

Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Bremia lactucae on lettuce,
Erysiphe cichoracearum and Sphaerotheca fuliginea on cucurbits,
Fusarium and Verticillium species on various plants,
Mycosphaerella species on cereals, bananas and peanuts,
Peronospora species on cabbage and onion plants,
Phatzopsora pachyrhizi and P. meibomiae on soy
Phytophthora infestans on potatoes and tomatoes,
Phytophthora capsici on peppers,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Pythium aphanidermatum on lawns,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (for example wood, paper, paint dispersions, fibers or fabrics) and in the protection of stored products.

In addition, the compounds of the formula I may also be used in crops which tolerate attack by insects or fungi owing to breeding, including genetic engineering methods.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted to the customary formulations, for example solutions, emulsions, suspensions, dusts, powders, pastes and granules. The application form depends on the particular intended use; it should in any case ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:

water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for broadcasting and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for Dilution with Water

### A) Water-Soluble Concentrates (SL)

10 parts by weight of a compound according to the invention are dissolved in water or in a water-soluble solvent. As an alternative, wetters or other auxiliaries are added. The active compound dissolves upon dilution with water.

### B) Dispersible Concentrates (DC)

20 parts by weight of a compound according to the invention are dissolved in cyclohexanone with addition of a dispersant, for example polyvinylpyrrolidone. Dilution with water gives a dispersion.

### C) Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D) Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E) Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

### F) Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

### G) Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

### 2. Products to be Applied Undiluted

### H) Dustable Powders (DP)

5 parts by weight of a compound according to the invention are ground finely and mixed intimately with 95% of finely divided kaolin. This gives a dustable product.

### I) Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

### J) ULV Solutions (UL)

10 parts by weight of a compound according to the invention are dissolved in an organic solvent, for example xylene. This gives a product to be applied undiluted.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should always ensure the finest possible dispersion of the active compounds according to the invention.

Aqueous application forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if appropriate also not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

Accordingly, the present invention also provides a combination of a compound of the formula I and at least one further fungicide, an insecticide and/or herbicide.

The following list of fungicides, with which the compounds according to the invention can be used in conjunction, is intended to illustrate the possible combinations but does not limit them:
acylalanines, such as benalaxyl, metalaxyl, ofurace or oxadixyl;
amine derivatives, such as aldimorph, dodine, dodemorph, fenpropimorph, fenpropidin, guazatine, iminoctadine, spiroxamine or tridemorph;
anilinopyrimidines, such as pyrimethanil, mepanipyrim or cyprodinyl;
antibiotics, such as cycloheximide, griseofulvin, kasugamycin, natamycin, polyoxin or streptomycin;
azoles, such as bitertanol, bromoconazole, cyproconazole, difenoconazole, dinitroconazole, enilconazole, epoxiconazole, fenbuconazole, fluquinconazole, flusilazole, flutriafole, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole or triticonazole;
dicarboximides, such as iprodione, myclozolin, procymidone or vinclozolin;
dithiocarbamates, such as ferbam, nabam, maneb, mancozeb, metam, metiram, propineb, polycarbamate, thiram, ziram or zineb;
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, penthiopyrad, picobenzamide, probenazole, proquinazid, pyrifenox, pyroquilon, quinoxyfen, silthiofam, thiabendazole, thifluzamide, thiophanate-methyl, tiadinil, tricyclazole, triforine, 5-chloro-7-(4-methylpiperidin-1-yl)-6-(2,4,6-trifluorophenyl)-[1,2,4]triazolo[1,5-a]pyrimidine;
copper fungicides, such as Bordeaux mixture, copper acetate, copper oxychloride or basic copper sulfate;
nitrophenyl derivatives, such as binapacryl, dinocap, dinobuton or nitrophthal-isopropyl;
phenylpyrroles, such as fenpiclonil or fludioxonil;
sulfur;
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, phosphorus acid, propamocarb, phthalide, tolclofos-methyl, quintozene or zoxamide;
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin;
sulfenic acid derivatives, such as captafol, captan, dichlofluanid, folpet or tolylfluanid;
cinnamides and analogous compounds, such as dimethomorph, flumetover or flumorph.

### SYNTHESIS EXAMPLES

### Example A

### 4-Chloro-6-isopropoxy-2-(pyrazol-1-yl)-5-(2,4,6-trifluorophenyl)pyrimidine (Table, No. 5-1a-A-6)

### Aa) 4-Chloro-6-isopropoxy-2-methylthio-5-(2,4,6-trifluorophenyl)pyrimidine

Under an atmosphere of nitrogen and at room temperature, 1.18 g (29.5 mmol) of 60% sodium hydride were added with stirring to 80 ml of isopropanol, and the mixture was stirred for 30 min. A solution of 8.0 g (24.6 mmol) 4,6-dichloro-2-methylthio-5-(2,4,6-trifluorophenyl)pyrimidine in 40 ml isopropanol was added dropwise to this mixture, and the mixture was stirred at room temperature overnight. After concentration under reduced pressure at 40° C., 100 ml of water and 150 ml of methylene chloride were added, the pH was adjusted to 8 using 20 ml of ammonium chloride solution, and the organic phase was separated off and extracted twice with in each case 100 ml of methylene chloride. The combined extracts were washed twice with in each case 100 ml of water, dried over sodium sulfate and concentrated under reduced pressure. The crude product was purified by medium pressure chromatography on RP material using acetonitrile/water (70:30). Yield 3.65 g. ¹H-NMR (CDCl₃) δ=1.27 (d); 2.60 (s); 5.41 (m); 6.75 (t).

### Ab) 4-Chloro-6-isopropoxy-2-methylsulfonyl-5-(2,4,6-trifluorophenyl)-pyrimidine

At room temperature, 71 mg of sodium tungstate were added to 1.50 g (4.30 mmol) of 4-chloro-6-isopropoxy-2-methylthio-5-(2,4,6-trifluorophenyl)pyrimidine dissolved in 20 ml of glacial acetic acid, and 0.98 g (8.6 mmol) of 30% strength hydrogen peroxide were then added dropwise. The mixture was stirred at room temperature overnight, another 100 mg (0.86 mmol) of 30% strength hydrogen peroxide were added, the mixture was stirred at room temperature for 1 d, added to 100 ml of ice-water and extracted four times with in each case 50 ml of methylene chloride, and the extract was washed twice with in each case 50 ml of sodium bicarbonate solution and once with 50 ml of sodium chloride solution, dried over sodium sulfate and concentrated under reduced pressure. Yield 1.60 g. ¹H-NMR (CDCl₃) δ=1.33 (d); 3.40 (s); 5.53 (m); 6.82 (t).

### Ac) 4-Chloro-6-isopropoxy-2-(pyrazol-1-yl)-5-(2,4,6-trifluorophenyl)-pyrimidine

At 0-5° C., 74 mg (1.84 mmol) of 60% sodium hydride were added to 125 mg (1.84 mmol) of pyrazole in 8 ml of dimethylformamide, and the mixture as stirred at this temperature for 1 h. Under an atmosphere of nitrogen, this solution was added dropwise over a period of 20 min to a solution of 700 mg (1.84 mmol) of 4-chloro-6-isopropoxy-2-methylsulfonyl-5-(2,4,6-trifluorophenyl)pyrimidine in 6 ml of dimethylformamide, and the mixture was allowed to warm to room temperature and stirred overnight. After concentration under reduced pressure, the residue was taken up in 60 ml of water and 60 ml of methylene chloride, extracted three times with in each case 60 ml of methylene chloride, dried over sodium sulfate and concentrated under reduced pressure. The crude product was purified by silica gel chromatography using cyclohexane/ethyl acetate. Yield 440 mg of a solid of m.p. 106-107° C. ¹H-NMR (CDCl₃) δ=1.33 (d); 5.56 (m); 6.50 (m); 6.78 (t); 7.85 (m); 8.55 (m).

### Example B

### O-Methyl (4-chloro-6-isopropoxy-5-(2,4,6-trifluorophenyl)pyrimidin-2-yl)carbamidoxime (Table, No. 5-1g-A-6)

### Ba) 4-Chloro-2-cyano-6-isopropoxy-5-(2,4,6-trifluorophenyl)pyrimidine

700 mg (10.8 mmol) of potassium cyanide were added to 2.05 g (5.39 mmol) of 4-chloro-6-isopropoxy-2-methylsulfonyl-5-(2,4,6-trifluorophenyl)pyrimidine in 30 ml of acetonitrile, the mixture was stirred at room temperature for 30 h and concentrated under reduced pressure, the residue was taken up in 20 ml of water and 60 ml of methyl tert-butyl ether, washed twice with in each case 20 ml of water, dried over sodium sulfate and concentrated under reduced pressure. Yield 1.60 g of a solid of m.p. 32° C. ¹H-NMR (CDCl₃) δ=1.30 (d); 5.45 (m); 6.82 (t).

### Bb) (4-Chloro-6-isopropoxy-5-(2,4,6-trifluorophenyl)pyrimidin-2-yl)carbamidoxime

300 mg (0.92 mmol) of 4-chloro-2-cyano-6-isopropoxy-5-(2,4,6-trifluorophenyl)-pyrimidine, 83 mg (1.20 mmol) of hydroxylamine hydrochloride and 62 mg (0.74 mmol) of sodium bicarbonate in 4.5 ml of ethanol and 1.5 ml of water were stirred at room temperature overnight, and concentrated under reduced pressure, 10 ml of water were added, the mixture was extracted three times with in each case 20 ml methyl tert-butyl ether and the extracts were dried over sodium sulfate and concentrated under reduced pressure. The crude product was purified by silica gel chromatography using cyclohexane/ethyl acetate. Yield 300 mg of a solid of m.p. 151-153° C. ¹H-NMR (CDCl₃) δ=1.30 (d); 5.50 (m and br.); 6.77 (t).

### Bc) O-Methyl (4-chloro-6-isopropoxy-5-(2,4,6-trifluorophenyl)pyrimidin-2-yl)carbamidoxime

400 mg (1.11 mmol) of (4-chloro-6-isopropoxy-5-(2,4,6-trifluorophenyl)pyrimidin-2-yl)carbamidoxime and 190 mg (1.33 mmol) of methyl iodide, dissolved in 10 ml of dimethylformamide, were cooled to −20° C., and 150 mg (1.33 mmol) of potassium tert-butoxide were added. The mixture was allowed to warm to room temperature, stirred overnight, added to 40 ml of sodium dihydrogenphosphate solution and extracted four times with in each case 20 ml methyl tert-butyl ether, and the extracts were washed twice with in each case 20 ml of sodium dihydrogenphosphate solution and once with sodium chloride solution, dried over sodium sulfate and concentrated under reduced pressure. The crude product was purified by silica gel chromatography using cyclohexane/ethyl acetate. Yield 140 mg of a solid of m.p. 82-83° C. ¹H-NMR (CDCl₃) δ=1.30 (d); 4.05 (s); 5.42 (br.); 5.50 (m); 6.77 (t).

Particularly preferred compounds of the present invention are listed in table B.

TABLE B
active compounds

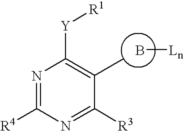

| No. | Y | R¹ | R³ | R⁴ | 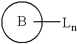 | phys. data |
|---|---|---|---|---|---|---|
| 1) | O | —CH(CH₃)₂ | Cl | pyrazol-1-yl | 2,4,6-trifluorophenyl | m.p. 106-107° C. |
| 2) | O | —CH(CH₃)₂ | Cl | —C(═NOH)NH₂ | 2,4,6-trifluorophenyl | m.p. 151-153° C. |
| 3) | O | —CH(CH₃)₂ | Cl | —C(═NOCH₃)NH₂ | 2,4,6-trifluorophenyl | ¹H-NMR (CDCl₃) δ = 1.30 |
| | | | | | | (d); 4.05 (s); 5.42 (br); 5.50 |
| | | | | | | (m); 6.77 (t). |
| 4) | O | —C(CH₃)₃ | Cl | pyridin-2-yl | 2-chloro-6- | ¹H-NMR (CDCl₃) δ = 1.66 |
| | | | | | fluorophenyl | (s), 7.06 (t), 7.30 (m), 7.35 |
| | | | | | | (m), 7.40 (m), 7.80 (t), 8.37 |
| | | | | | | (d), 8.80 (d). |
| 5) | O | —CH(CH₃)₂ | Cl | —CN | 2,4,6-trifluorophenyl | ¹H-NMR (CDCl₃) δ = 1.30 |
| | | | | | | (d); 5.45 (m); 6.82 (t). |
| 6) | O | —CH(CH₃)(C₂H₅) | Cl | pyrazol-1-yl | 2,4,6-trifluorophenyl | m.p. 139-140° C. |
| 7) | O | —CH₂CH₂CH₂CH₃ | —OCH₂CH₂CH₂CH₃ | pyridin-2-yl | 2,4,6-trifluorophenyl | ¹H-NMR (CDCl₃): δ = |
| | | | | | | 0.85 (t, 3H); 0.95 (t, 3H); |
| | | | | | | 1.40 (m, 4H); 2.70 (m, 2H); |
| | | | | | | 2.72 (m, 2H); 4.60 (m, 2H); |
| | | | | | | 4.65 (m, 2H); 6.80 (t, 2H); |
| | | | | | | 7.22 (t, 1H); 7.82 (t, 1H); |
| | | | | | | 8.50 (d, 1H); 8.90 (d, 1H) |
| 8) | O | —CH₂CH₂CH₂CH₃ | Cl | pyridin-2-yl | 2,4,6-trifluorophenyl | ¹H-NMR (CDCl₃): δ = |
| | | | | | | 0.85 (t, 3H); 1.40 (m, 2H); |
| | | | | | | 2.70 (m, 2H); 4.60 (m, 2H); |
| | | | | | | 6.80 (t, 2H); 7.22 (t, 1H); |
| | | | | | | 7.82 (t, 1H); 8.50 (d, 1H); |
| | | | | | | 8.90 (d, 1H) |
| 9) | O | —CH₂CH₂CH₃ | —OCH₂CH₂CH₃ | pyridin-2-yl | 2,4,6-trifluorophenyl | m.p. 103-104° C. |
| 10) | O | —CH(CH₃)₂ | Cl | pyridin-2-yl | 2,4,6-trifluorophenyl | m.p. 90-92° C. |
| 11) | O | —C(CH₃)₃ | Cl | pyridin-2-yl | 2,4,6-trifluorophenyl | ¹H-NMR (CDCl₃): δ = |
| | | | | | | 1.60 (s, 9H); 6.78 (t, 2H); |
| | | | | | | 7.42 (m, 1H); 7.86 (t, 1H); |
| | | | | | | 8.48 (d, 1H); 8.86 (d, 1H) |
| 12) | O | —CH₂CH₂CH₃ | Cl | pyridin-2-yl | 2,4,6-trifluorophenyl | ¹H-NMR (CDCl₃): δ = |
| | | | | | | 0.95 (m, 3H); 1.78 (m, 2H); |
| | | | | | | 4.56 (m, 2H); 6.80 (m, 2H); |
| | | | | | | 7.46 (t, 1H); 7.90 (t, 1H); |
| | | | | | | 8.50 (d, 1H); 8.86 (d, 1H) |
| 13) | O | —C₂H₅ | Cl | pyridin-2-yl | 2,4,6-trifluorophenyl | ¹H-NMR (CDCl₃): δ = |
| | | | | | | 1.38 (t, 3H); 4.64 (m, 2H); |
| | | | | | | 6.80 (m, 2H); 7.41 (t, 1H); |
| | | | | | | 7.90 (t, 1H); 8.50 (d, 1H); |
| | | | | | | 8.86 (d,1H) |
| 14) | O | —CH₂CH₂CH₃ | Cl | pyrazol-1-yl | 2,4,6-trifluorophenyl | ¹H-NMR (CDCl₃): δ = |
| | | | | | | 0.92 (t, 3H); 1.72 (m, 2H); |
| | | | | | | 4.50 (t, 2H); 6.50 (s, 1H); |
| | | | | | | 6.80 (t, 2H); 7.86 (s, 1H); |
| | | | | | | 8.58 (s, 1H) |
| 15) | O | —CH(CH₃)(C₂H₅) | Cl | —CN | 2,4,6-trifluorophenyl | ¹H-NMR (CDCl₃): δ = |
| | | | | | | 0.87 (t); 1.33 (d); 1.63 (m); |
| | | | | | | 5.35 (m); 6.83 (m). |
| 16) | O | —CH(CH₃)(C₂H₅) | Cl | —C(═NOH)NH₂ | 2,4,6-trifluorophenyl | m.p. 66-68° C. |
| 17) | O | —CH(CH₃)CH(CH₃)₂ | Cl | pyrazol-1-yl | 2,4,6-triflurophenyl | m.p. 96-98° C. |
| 18) | O | —CH(CH₃)₂ | Cl | —C(═NOCH₃)NH(CO—CH₃) | 2,4,6-trifluorophenyl | ¹H-NMR (CDCl₃): δ = |
| | | | | | | 1.27 (d); 2.18 (s); 4.05 (s); |
| | | | | | | 5.40 (m); 6.80 (t); 8.00 (s). |
| 19) | S | —CH(CH₃)₂ | Cl | [1,2,4]triazol-1-yl | 2,4,6-trifluorophenyl | ¹H-NMR (CDCl₃): δ = |
| | | | | | | 1.50 (d); 4.00 (m); 6.75 (t); |
| | | | | | | 7.83 (s); 9.15 (s). |
| 20) | O | —CH(CH₃)₂ | CH₃ | —CN | 2,4-difluorophenyl | m.p. 77-78° C. |
| 21) | O | —CH(CH₃)₂ | Cl | —CN | 2-chloro-4- | m.p. 66-67° C. |
| | | | | | fluorophenyl | |
| 22) | O | —CH(CH₃)₂ | CH₃ | —(C═NOCH₃)NH₂ | 2,4-difluorophenyl | m.p. 108-110° C. |
| 23) | O | —CH(CH₃)₂ | Cl | [1,2,4]triazol-1-yl | 2-chloro-4- | ¹H-NMR (CDCl₃): δ = |
| | | | | | fluorophenyl | 1.30 (d); 5.55 (m); 7.13 |
| | | | | | | (m); 7.25 (m); 8.20 (m); |
| | | | | | | 9.20 (m). |
| 24) | O | —CH(CH₃)₂ | Cl | —C(═NOH)NH₂ | 2-chloro-4- | m.p. 188-189° C. |
| | | | | | fluorophenyl | |
| 25) | O | —CH(CH₃)₂ | Cl | —C(O)NH₂ | 2-chloro-4- | m.p. 174-176° C. |
| | | | | | fluorophenyl | |
| 26) | O | —CH(CH₃)₂ | Cl | —C(═NOCH₃)NH₂ | 2-chloro-4- | m.p. 147-149° C. |
| | | | | | fluorophenyl | |
| 27) | O | —CH(CH₃)₂ | CH₃ | —C(═NOH)NH₂ | 2,4-difluorophenyl | m.p. 174-175° C. |
| 28) | O | —CH(C₂H₅)₂ | Cl | —CN | 2,4,6-trifluorophenyl | ¹H-NMR (CDCl₃): δ = |
| | | | | | | 0.84 (m, 6H); 1.62 (m, 4H); |
| | | | | | | 5.25 (m, 1H); 6.70 (m, 2H) |
| 29) | O | —CH(C₂H₅)(CH₂CH₂CH₃) | Cl | pyrazol-1-yl | 2,4,6-trifluorophenyl | ¹H-NMR (CDCl₃): δ = |
| | | | | | | 0.88 (m, 6H); 1.30 (m, 2H); |
| | | | | | | 1.60 (m, 2H); 1.70 (m, 2H); |
| | | | | | | 5.40 (m, 1H); 6.50 (s, 1H); |
| | | | | | | 6.78 (m, 2H); 7.88 (s, 1H); |
| | | | | | | 8.52 (s, 1H) |
| 30) | O | —CH₂CH(CH₃)₂ | Cl | pyrazol-1-yl | 2,4,6-trifluorophenyl | ¹H-NMR (CDCl₃): δ = |
| | | | | | | 0.92 (d, 6H); 2.00 (m, 1H); |
| | | | | | | 4.30 (d, 2H); 6.50 (s, 1H); |
| | | | | | | 6.80 (m, 2H); 7.90 (s, 1H); |
| | | | | | | 8.58 (s, 1H) |
| 31) | O | —CH(CH₃)(C₂H₅) | Cl | —NHCH₃ | 2,4,6-trifluorophenyl | m.p. 66-68° C. |
| 32) | O | —CH₂C(CH₃)₃ | Cl | pyrazol-1-yl | 2,4,6-trifluorophenyl | ¹H-NMR (CDCl₃): δ = |
| | | | | | | 0.90 (s, 9H); 4.19 (s, 2H); |
| | | | | | | 6.50 (s, 1H); 6.80 (m, 2H); |
| | | | | | | 7.88 (s, 1H); 8.58 (s, 1H) |
| 33) | O | —CH(CH₃(CH₂CH₂CH₃) | Cl | pyrazol-1-yl | 2,4,6-trifluorophenyl | m.p. 110-112° C. |
| 34) | O | —CH(CH₃)CH₂CH(CH₃)₂ | Cl | pyrazol-1-yl | 2,4,6-trifluorophenyl | m.p. 124-125° C. |
| 35) | O | cyclohexyl | Cl | pyrazol-1-yl | 2,4,6-trifluorophenyl | m.p. 104-105° C. |
| 36) | O | cyclopentyl | Cl | pyrazol-1-yl | 2,4,6-trifluorophenyl | m.p. 81-83° C. |
| 37) | O | —CH(C₂H₅)₂ | Cl | pyrazol-1-yl | 2,4,6-trifluorophenyl | ¹H-NMR (CDCl₃): δ = |
| | | | | | | 0.85 (t, 6H); 1.64 (m, 4H); |
| | | | | | | 5.32 (m, 1H); 6.50 (s, 1H); |
| | | | | | | 6.80 (m, 2H); 7.88 (s, 1H); |
| | | | | | | 8.52 (s, 1H) |
| 38) | O | 4-methylcyclohexyl | Cl | pyrazol-1-yl | 2,4,6-trifluorophenyl | ¹H-NMR (CDCl₃): δ = |
| | | | | | | 0.80 (d, 3H); 0.92 (d, 1H); |
| | | | | | | 1.00 (m, 2H); 1.45 (m, 2H); |
| | | | | | | 1.58 (t, 2H); 1.98 (d, 2H); |
| | | | | | | 5.55 (s, 1H); 6.52 (s, 1H); |
| | | | | | | 6.78 (m, 2H); 7.88 (s, 1H); |
| | | | | | | 8.58 (s, 1H) |
| 39) | O | —CH(CH₃)CH₂C(CH₃)₃ | Cl | pyrazol-1-yl | 2,4,6-trifluorophenyl | ¹H-NMR (CDCl₃): δ = |
| | | | | | | 0.80 (s, 9H); 1.32 (d, 3H); |
| | | | | | | 1.60 (m, 2H); 5.62 (m, 1H); |
| | | | | | | 6.56 (s, 1H); 6.78 (m, 2H); |
| | | | | | | 7.88 (s, 1H); 8.58 (s, 1H) |
| 40) | O | —CH(CH₃)₂ | CH₃ | —C(═NOCH₃)NH₂ | 2,4-dichlorophenyl | ¹H-NMR (CDCl₃): δ = |
| | | | | | | 1.27 (m); 2.28 (s); 4.05 (s); |
| | | | | | | 5.02 (m); 5.45 (s); 7,10 (d); |
| | | | | | | 7.35 (m); 7.54 (d). |
| 41) | O | —CH₂CF₃ | Cl | —CN | 2,4,6-trifluorophenyl | ¹H-NMR (CDCl₃): δ = |
| | | | | | | 4.87 (q); 6.85 (m). |
| 42) | O | —CH₂CF₃ | Cl | [1,2,4]triazol-1-yl | 2,4,6-trifluorophenyl | m.p.123-124° C. |
| 43) | O | —CH(CH₃)(CH₂CH₂CH₃) | Cl | —C(═NOH)NH₂ | 2,4,6-trifluorophenyl | ¹H-NMR (CDCl₃): δ = |
| | | | | | | 0.85 (t, 3H); 1.30 (d, 5H); |
| | | | | | | 1.55 (m, 1H); 1.65 (m, 1H); |
| | | | | | | 4.45 (m, 1H); 5.58 (s, 2H); |
| | | | | | | 6.78 (m, 2H) |
| 44) | O | —CH₂CH(CH₃)₂ | Cl | —C(═NOCH₃)NH₂ | 2,4,6-trifluorophenyl | ¹H-NMR (CDCl₃): δ = |
| | | | | | | 0.88 (m, 6H); 1.88 (m, 1H); |
| | | | | | | 4.08 (m, 3H); 4.24 (d, 2H); |
| | | | | | | 5.46 (s, 2H); 6.80 (m, 2H) |
| 45) | O | —CH(CH₃)₂ | —SCH₃ | —C(═NOCH₃)NH₂ | 2,4,6-trifluorophenyl | m.p. 78-82° C. |
| 46) | S | —CH(CH₃)₂ | —CH₃ | [1,2,4]triazol-1-yl | 2,4,6-trifluorophenyl | m.p. 133-135° C. |
| 47) | O | —CH(CH₃)₂ | Cl | [1,2,4]triazol-1-yl | 2,4,6-trifluorophenyl | ¹H-NMR (CDCl₃): δ = |
| | | | | | | 1.35 (d); 5.57 (m); 6.83 (t); |
| | | | | | | 8.20 (s); 9.21 (s). |
| 48) | O | —CH(CH₃)₂ | —CH₃ | —CN | 2-chloro-4- | m.p. 76-79° C. |
| | | | | | fluorophenyl | |
| 49) | O | —CH(CH₃)₂ | —CH₃ | —CN | 2-chloro-4- | ¹H-NMR (CDCl₃): δ = |
| | | | | | methoxyphenyl | 1.25 (m); 2.25 (s); 3.87 (s); |
| | | | | | | 5.38 (m); 6.92 (m); 7.08 |
| | | | | | | (m). |
| 50) | O | —CH(CH₃)₂ | —C₂H₅ | —CN | 2-chloro-4- | m.p. 77-83° C. |
| | | | | | fluorophenyl | |
| 51) | O | —CH(CH₃)₂ | —CH₃ | —C(═NOCH₃)NH₂ | 2-chloro-4- | m.p. 142-144° C. |
| | | | | | fluorophenyl | |
| 52) | O | —CH(CH₃)₂ | —CH₃ | —C(═NOH)NH₂ | 2-chloro-4- | m.p. 206-218° C. |
| | | | | | fluorophenyl | |
| 53) | O | —CH(CH₃)₂ | —CH₃ | —C(═O)NH₂ | 2-chloro-4- | m.p. 153-176° C. |
| | | | | | fluorophenyl | |
| 54) | O | —CH(CH₃)₂ | —CH₃ | —C(═NH)NH₂ | 2-chloro-4- | m.p. 170-177° C. |
| | | | | | fluorophenyl | |
| 55) | O | —CH(CH₃)₂ | —CH₃ | —C(═NOCH₃)NH₂ | 2-chloro-4- | m.p. 103-110° C. |
| | | | | | methoxyphenyl | |
| 56) | O | —CH(CH₃)₂ | —CH₃ | —C(═NOH)NH₂ | 2-chloro-4- | m.p. 184-195° C. |
| | | | | | methoxyphenyl | |
| 57) | O | —CH(CH₃)₂ | —CH₃ | —C(═O)NH₂ | 2-chloro-4- | m.p. 172-185° C. |
| | | | | | methoxyphenyl | |
| 58) | O | —CH(CH₃)₂ | —CH₃ | —C(═NH)NH₂ | 2-chloro-4-methoxy- | m.p. 154-165° C. |
| | | | | | phenyl | |
| 59) | O | —CH(CH₃)₂ | —C₂H₅ | —C(═NOCH₃)NH₂ | 2-chloro-4- | m.p. 118-122° C. |
| | | | | | fluorophenyl | |
| 60) | O | —CH(CH₃)₂ | —C₂H₅ | —C(═NOH)NH₂ | 2-chloro-4- | m.p. 219-226° C. |
| | | | | | fluorophenyl | |
| 61) | O | —CH(CH₃)₂ | —C₂H₅ | —C(═O)NH₂ | 2-chloro-4- | m.p. 91-110° C. |
| | | | | | fluorophenyl | |
| 62) | O | —CH(CH₃)₂ | —C₂H₅ | —C(═NH)NH₂ | 2-chloro-4- | m.p. 167-170° C. |
| | | | | | fluorophenyl | |
| 63) | O | —CH(CH₃)(CH₂CH₂CH₃) | Cl | —CN | 2,4,6-trifluorophenyl | ¹H-NMR (CDCl₃): δ = |
| | | | | | | 0.90 (m, 3H); 1.28 (d, 5H); |
| | | | | | | 1.56 (m, 1H); 1.64 (m, 1H); |
| | | | | | | 5.40 (m, 1H); 6.80 (m, 2H) |
| 64) | O | —CH(CH₃)CH₂CH(CH₃)₂ | —OCH₃ | —C(═NOCH₃)NH₂ | 2,4,6-trifluorophenyl | ¹H-NMR (CDCl₃): δ = |
| | | | | | | 0.84 (m, 9H); 1.28 (m, 5H); |
| | | | | | | 1.56 (m, 1H); 1.68 (m, 1H); |
| | | | | | | 4.02 (s, 3H); 5.42 (s, 2H); |
| | | | | | | 6.78 (m, 2H) |
| 65) | O | cyclohexyl | —OCH₃ | —C(═NOCH₃)NH₂ | 2,4,6-trifluorophenyl | ¹H-NMR (CDCl₃): δ = |
| | | | | | | 0.90 (m, 2H); 1.38 (s, 3H); |
| | | | | | | 1.50 (m, 2H); 1.65 (m, 2H); |
| | | | | | | 1.88 (m, 2H); 4.02 (s, 3H); |
| | | | | | | 5.20 (m, 1H); 5.42 (s, 2H); |
| | | | | | | 6.72 (m, 2H) |
| 66) | O | cyclopentyl | —OCH₃ | —C(═NOCH₃)NH₂ | 2,4,6-trifluorophenyl | ¹H-NMR (CDCl₃): δ = |
| | | | | | | 1.25 (s, 3H); 1.58 (m, 2H); |
| | | | | | | 1.68 (m, 2H); 1.76 (m, 2H); |
| | | | | | | 1.92 (m, 2H); 4.02 (s, 3H); |
| | | | | | | 5.45 (s, 2H); 5.60 (m, 1H); |
| | | | | | | 6.72 (m, 2H) |
| 67) | O | —CH(CH₃)(C₂H₅) | Cl | —C(═NOCH₃)NH₂ | 2,4,6-trifluorophenyl | m.p. 88-92° C. |
| 68) | O | —CH(CH₃)CH(CH₃)₂ | Cl | —C(═NOH)NH₂ | 2,4,6-trifluorophenyl | m.p. 140-141° C. |
| 69) | O | —CH(CH₃)CH(CH₃)₂ | Cl | —C(═NOCH₃)NH₂ | 2,4,6-trifluorophenyl | m.p. 96-101° C. |

### Examples of the Action Against Harmful Fungi

The fungicidal action of the compounds of the formula I was demonstrated by the following experiments:

The active compounds were prepared separately as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### USE EXAMPLES

### Use Example 1

### Activity Against Early Blight of Tomato Caused by Alternaria solani

Leaves of potted plants of the cultivar “Goldene Königin” were sprayed to runoff point with an aqueous suspension having the concentration of active compounds stated below. The next day, the leaves were inoculated with an aqueous spore suspension of Alternaria solani in 2% biomalt solution having a density of 0.17×10⁶ spores/ml. The plants were then placed in a water-vapor-saturated chamber at temperatures of between 20 and 22° C. After 5 days, the disease on the leaves of the untreated, but infected control plants had developed to such an extent that the infection could be determined visually in %.

The leaf areas which had been treated with an application rate of 250 ppm of compounds 1), 3) or 4) of Table B showed only little infection, whereas the untreated leaf areas were 90% infected.

The plants which had been treated with 250 ppm of the compounds 6), 7), 8), 10), 12), 13), 14), 15), 28), 29), 30), 32), 33), 35), 36), 38), 39), 40), 43), 45), 46), 47), 51), 53), 55), 56) or 57) and with 63 ppm of the compounds 17), 22), 23), 25), 26) or 42), too, showed an infection of at most 20%, whereas the untreated leaf areas were 90% infected.

### Use Example 2

### Activity Against Gray Mold on Bell Pepper Leaves Caused by Botrytis cinerea, Protective Application

Bell pepper seedlings of the cultivar “Neusiedler Ideal Elite” were, after 2-3 leaves were well-developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

The leaf areas which had been treated with an application rate of 250 ppm of compounds 1), 3) or 4) of Table B showed only little infection, whereas the untreated leaf areas were 90% infected.

The plants which had been treated with 250 ppm of the compounds 7), 10), 12), 13), 14), 20), 21), 24), 27), 30), 32), 36), 43), 47), 48), 49), 51), 52), 53), 55), 56), 57), 67), 68), or 69) and 63 ppm of the compounds 16), 17), 22), 23), 25), 26) or 42), too, showed an infection of at most 20%, whereas the untreated leaf areas were 90% infected.

### Use Example 3

### Activity Against Net Blotch of Barley Caused by Pyrenophora teres, 1 Day Protective Application

Leaves of potted barley seedlings of the cultivar “Hanna” were sprayed to runoff point with an aqueous suspension having the concentration of active compounds stated below. 24 hours after the spray coating had dried on, the test plants were inoculated with an aqueous spore suspension of Pyrenophora [syn. Drechslera] teres, the net blotch pathogen. The test plants were then placed in a greenhouse at temperatures between 20 and 24° C. and 95 to 100% relative atmospheric humidity. After 6 days, the extent of the development of the disease was determined visually in % infection of the entire leaf area.

The leaf areas which had been treated with an application rate of 250 ppm of compounds 1), 3) or 4) of Table B showed only little infection, whereas the untreated leaf areas were 90% infected.

The plants which had been treated with 250 ppm of the compounds 8), 24), 35), 38), 46), 68), or 69), too, showed an infection of at most 20%, whereas the untreated leaf areas were 90% infected.

1. A 2-substituted pyrimidine of the formula I 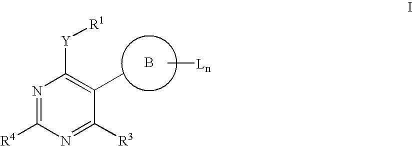
in which the indices and the substituents are as defined below:
Y is —O— or —S—;
R¹ is C₁-C₈-alkyl, C₂-C₈-alkenyl, C₂-C₈-alkynyl, C₃-C₆-cycloalkyl, C₃-C₆-halocycloalkyl, di-C₁-C₈-alkylamino, C₁-C₈-alkylamino, where R¹ for its part may be partially or fully halogenated or may carry one to four groups R²:
R² is cyano, C₁-C₆-alkyl, C₃-C₆-cycloalkyl, C₄-C₆-cycloalkenyl, hydroxyl, C₁-C₆-alkoxy, C₂-C₈-alkenyloxy, C₂-C₈-alkynyloxy, C₃-C₆-cycloalkyloxy, C₄-C₆-cycloalkenyloxy, C₁-C₆-alkylthio, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A or phenyl, where the phenyl moiety may carry one to three radicals selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N-OA), N(A′)A;
or —CH₂—Si(C₁-C₆-alkyl)₂;
R³ is halogen, cyano, azido, C₁-C₄-alkyl, C₂-C₄-alkenyl, C₂-C₄-alkynyl, C₃-C₆-cycloalkyl, C₁-C₄-alkoxy, C₃-C₄-alkenyloxy, C₃-C₄-alkynyloxy, C₁-C₆-alkylthio, di(C₁-C₆-alkyl)amino or C₁-C₆-alkylamino, where the alkyl, alkenyl and alkynyl radicals of R³ may be substituted by halogen, cyano, nitro, C₁-C₂-alkoxy or C₁-C₄-alkoxycarbonyl;
R⁴ is a five- or six-membered saturated, partially unsaturated or aromatic mono- or bicyclic heterocycle which comprises one to four heteroatoms from the group consisting of O, N and S which for its part may be partially or fully halogenated or may carry one to four groups R^{u}:
R^{u} is cyano, C₁-C₈-alkyl, C₂-C₈-alkenyl, C₂-C₈-alkynyl, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A, where m, A, A′, A″ are as defined above;
R⁴ may furthermore be:
cyano, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c},
ON(═CR^{a}R^{b}), O—C(=Z)R^{a},
NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}, where
Z is O, S, NR^{a}, NOR^{a} or N—NR^{z}R^{c};
R^{a}, R^{b}, R^{c} independently of one another are hydrogen, C₁-C₆-alkyl, C₂-C₆-alkenyl, C₂-C₆-alkynyl, C₃-C₆-cycloalkyl or C₄-C₆-cycloalkenyl;
R^{b′} has the same meanings as R^{b}, except for hydrogen;
R^{z} has the same meanings as R^{a} and may additionally be —CO—R^{a};
where the aliphatic or alicyclic groups of the radical definitions of R^{a}, R^{b}, R^{c} or R^{z} for their part may be partially or fully halogenated or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, and where two of the radicals R^{a}, R^{b}, R^{c} or R^{z} together with the atoms, to which they are attached, may form a five- or six-membered saturated, partially unsaturated or aromatic heterocycle which comprises one to four heteroatoms from the group consisting of O, N and S;
\textcircled{B} is a five- or six-membered hetaryl which comprises 1 to 3 heteroatoms selected from the group consisting of O, N and S or is phenyl;
n is an integer from 1 to 5;
L is halogen, cyano, cyanato (OCN), C₁-C₈-alkyl, C₂-C₈-alkenyl, C₂-C₈-alkynyl, C₁-C₆-alkoxy, C₂-C₈-alkenyloxy, C₂-C₈-alkynyloxy, C₃-C₆-cycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A,
m is 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by nitro, cyanato, cyano or C₁-C₄-alkoxy; or A and A′ together with the atoms to which they are attached are a five- or six-membered saturated, partially unsaturated or aromatic heterocycle which comprises one to four heteroatoms from the group consisting of O, N and S;
where the aliphatic groups of the radical definitions of L for their part may be partially or fully halogenated or may carry one to four groups R^{L}:
R^{L} is cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N-OA), N(A′)A, N(A′)—C(═O)-A, N(A″)—C(═O)—N(A′)A, S(═O)ₘ-A, S(═O)ₘ, —O-A or S(═O)ₘ—N(A′)A.

2. The 2-substituted pyrimidine according to claim 1, in which B=phenyl and which corresponds to the formula I′ 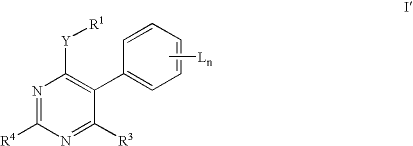
where
Y is —S— or —O—;
R¹ is C₁-C₆-alkyl, C₁-C₆-cycloalkyl, C₁-C₄-alkyl-C₁-C₆-cycloalkyl, di-C₁-C₄-alkyl-C₁-C₆-cycloalkyl, C₁-C₄-alkoxy-C₁-C₆-alkyl, C₂-C₆-alkenyl, C₂-C₆-alkynyl, C₁-C₆-haloalkyl, di-C₁-C₈-alkylamino, C₁-C₈-alkylamino, C₂-C₆-haloalkenyl or C₂-C₆-haloalkynyl;
R³ is halogen, cyano, C₁-C₄-alkyl, C₁-C₄-alkoxy or C₁-C₄-haloalkyl;
R⁴ is pyrrole, pyrazole, imidazole, 1,2,3-triazole, 1,2,4-triazole, tetrazole, oxazole, isoxazole, 1,3,4-oxadiazole, furan, thiophene, thiazole, isothiazole, pyridine, pyrimidine, pyrazine, pyridazine, 1,2,3-triazine, 1,2,4-triazine, 1-pyridin(1,2,-dihydro)-2-one or 1-pyrrolidone, where the heterocycle may be attached via C or N to the pyrimidine ring and may carry up to three substituents R^{u};
R^{u} is halogen, cyano, C₁-C₈-alkyl, C₁-C₆-alkoxy, —C(═O)-A, —C(═O)—O-A, —C(═O)—N(A′)A, C(A′)(═N-OA), N(A′)A, N(A′)-C(═O)-A,
or
cyano, C(═O)NR^{z}R^{b}, C(═O)OR^{a}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}N═CR^{c}R^{b}) or NR^{z}—OR^{a};
n is an integer from 1 to 3 where at least one substituent L is located in the ortho-position on the phenyl ring;
L is halogen, cyano, methyl, methoxy, —C(═O)—O-A, —C(═O)—N(A′)A, —C(═S)—N(A′)A, C(A′)(═N-OA), N(A′)A, N(A′)-C(═O)-A,
A,A′ independently of one another are hydrogen, C₁-C₆-alkyl, C₂-C₆-alkenyl, C₂-C₆-alkynyl, phenyl, where the organic radicals may be partially or fully halogenated or may be substituted by C₁-C₄-alkoxy; or A and A′ together with the atoms to which they are attached are a five- or six-membered saturated heterocycle which comprises one or two heteroatoms from the group consisting of O, N and S;
where the aliphatic groups of the radical definitions of L for their part may be partially or fully halogenated.

3. The 2-substituted pyrimidine according to claim 1, in which B=phenyl and which corresponds to the formula I′ 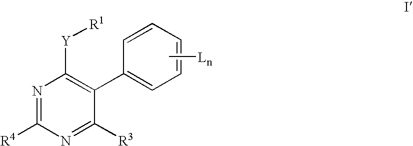
where
Y is —S— or —O—;
R¹ is C₁-C₆-alkyl, C₁-C₆-cycloalkyl, C₁-C₄-alkyl-C₁-C₆-cycloalkyl, di-C₁-C₄-alkyl-C₁-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl;
R³ is halogen, cyano, C₁-C₄-alkyl, C₁-C₄-alkoxy, halomethoxy or C₁-C₄-haloalkyl;
R⁴ is pyrazole, 1,2,3-triazole or 1,2,4-triazole, where the heterocycle is attached via N to the pyrimidine ring and may carry up to two substituents R^{u};
R^{u} is halogen, cyano, C₁-C₄-alkyl, C₁-C₄-alkoxy, —C(═O)—O-A, —C(═O)—N(A′)A, C(A′)(═N-OA), N(A′)A, N(A′)-C(═O)-A,
or
C(═O)NR^{z}R^{b}, C(═O)OR^{a}, C(═NOR^{a})NH₂, C(═NOR^{b})R^{a} or NR^{a}(C(═O)OR^{b}), where
R^{a}, R^{b}, R^{c} independently of one another are hydrogen, C₁-C₆-alkyl or C₃-C₆-cycloalkyl;
n is an integer from 1 to 3 where at least one substituent L is located in the ortho-position on the phenyl ring;
L is halogen, cyano, methyl, methoxy, —C(═O)—O-A, —C(═O)—N(A′)A, —C(═S)—N(A′)A, C(A′)(═N—OA), N(A′)A, N(A′)-C(═O)-A,
A,A′ independently of one another are hydrogen, C₁-C₆-alkyl, where the organic radicals may be partially or fully halogenated or substituted by C₁-C₄-alkoxy.

4. The 2-substituted pyrimidine according to claim 1 in which
R⁴ is 1-pyrazole or 1-[1,2,4]triazole.

5. The 2-substituted pyrimidine according to claim 1 in which
R⁴ is 2-pyridine, 3-pyridazine, 1-pyridin(1,2,-dihydro)-2-one or 1-pyrrolidone.

6. The 2-substituted pyrimidine according to claim 1 in which
R⁴ is 2-pyrimidine.

7. The 2-substituted pyrimidine according to claim 1 in which
R⁴ is C(=Z)OR^{a}, C(=Z)NR^{z}R^{b} or C(=Z)R^{a} and
Z is O, NR^{a} or NOR^{a}.

8. The 2-substituted pyrimidine according to claim 1 in which
Y is a group —O— and
R¹ is C₃-C₈-alkyl, C₃-C₈-alkenyl, C₃-C₈-alkynyl or C₃-C₆-haloalkyl branched in the α-position.

9. The 2-substituted pyrimidine according to claim 1 in which the group B substituted by Lₙ is phenyl and is shown by 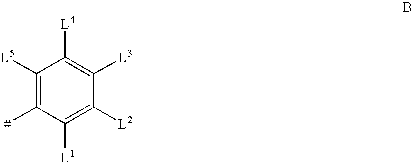
where # is the point of attachment to the pyrimidine skeleton and
L¹ is fluorine, chlorine, CH₃ or CF₃;
L², L⁴ independently of one another are hydrogen, CH₃ or fluorine;
L³ is hydrogen, fluorine, chlorine, cyano, nitro, CH₃, SCH₃, OCH₃, SO₂CH₃, NH—C(═O)CH₃, N(CH₃)—C(═O)CH₃, C(═S)NH₂ or COOCH₃ and
L⁵ is hydrogen, fluorine, chlorine or CH₃.

10. A pesticidal composition which comprises a solid or liquid carrier and a compound of the formula I according to claim 1.

11. A combination of a compound of the formula I according to claim 1 and at least one further fungicide, an insecticide and/or herbicide.

12. A method for controlling phytopathogenic harmful fungi which comprises treating the fungi or the materials, plants, the soil or the seeds to be protected against fungal attack with an effective amount of a compound of the formula I according to claim 1.

13. A method for controlling animal pests in agriculture which comprises treating the pests or the materials, plants, the soil or the seeds to be protected against them with an effective amount of a compound of the formula I according to claim 1.
